(19) United States
(12) Patent Application Publication
(10) Pub. No.: US 2018/0071284 A1
(43) Pub. Date: Mar. 15, 2018

(54) PREVENTING OR TREATING VIRAL INFECTION BY INHIBITION OF THE HISTONE METHYLTRANSFERASE EZH1 OR EZH2

(71) Applicant: The United States of America, as represented by the Secretary, Department of Health and Human Serv, Bethesda, MD (US)

(72) Inventors: Thomas M. Kristie, Silver Spring, MD (US); Jesse H. Arbuckle, Rockville, MD (US)

(73) Assignee: The United States of America, as represented by the Secretary, Department of Health and Human Serv, Bethesda, MD (US)

(21) Appl. No.: 15/800,515

(22) Filed: Nov. 1, 2017

Related U.S. Application Data
(63) Continuation-in-part of application No. PCT/US2016/030089, filed on Apr. 29, 2016.
(60) Provisional application No. 62/155,704, filed on May 1, 2015.

(51) Int. Cl. A61K 31/496; A61K 31/454; A61K 9/00

(52) U.S. Cl. CPC A61K 31/496; A61K 9/0014; A61K 31/454

(57) ABSTRACT

Disclosed are methods of preventing or treating a viral infection of a host, the method comprising administering to the host an effective amount of an inhibitor of the histone methyltransferase activity of EZH1 or EZH2. In one embodiment, the method comprises administering to the host an effective amount of a compound of Formula (I):

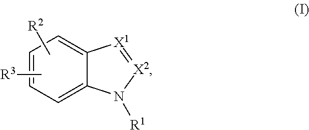

wherein X¹, X², R¹, R², and R³ are defined herein; or pharmaceutically acceptable salts, solvates, or stereoisomers thereof. In another embodiment, the present invention provides a method of inhibiting an EZH1 or EZH2 methyltransferase in a virus-infected host, the method comprising administering to the host an effective amount of a compound of Formula (I) as defined above. In another embodiment, the present invention provides a method of improving the therapeutic effect of a pharmaceutical composition, the method comprising adding to the pharmaceutical composition a compound of Formula (I) as defined above.

### CROSS-REFERENCE TO A RELATED APPLICATION

This application is a continuation-in-part of International Patent Application No. PCT/US2016/030089, filed Apr. 29, 2016, which claims the benefit of U.S. Provisional Patent Application No. 62/155,704, filed May 1, 2015, each of which is incorporated herein by reference in its entirety.

### STATEMENT REGARDING FEDERALLY SPONSORED RESEARCH AND DEVELOPMENT

This invention was made with Government support under project numbers ZIA AI000712 LVD and ZIA AI000711 LVD by the National Institutes of Health, National Institute of Allergy and Infectious Diseases. The Government has certain rights in the invention.

### BACKGROUND OF THE INVENTION

Herpes viral infections, including herpes simplex virus type 1 (HSV-1) and type 2 (HSV-2) infections, are common infections worldwide. These viruses establish lifelong persistent infections with cycles of lytic reactivation to produce recurrent diseases including oral and genital lesions, herpetic keratitis/blindness, congenital-developmental syndromes, and viral encephalitis. Additionally, infection with HSV-2 increases the rate of human immunodeficiency virus (HIV) transmission in coinfected individuals. Initial infection with Varicella Zoster virus (VZV) results in vesicular disseminated lesions (Chicken pox), generally in children, while reactivation produces shingles, a disease with painful lesions often resulting in long-term neuropathy. Cytomegalovirus is an additional herpesvirus which is the leading viral cause of birth defects (hearing loss) and is a complicating factor in immunocompromised individuals including individuals undergoing organ transplant.

DNA replication inhibitors are typically used to treat herpesvirus infections. However, these compounds do not completely suppress infection, viral shedding, reactivation from latency, and the inflammation that contributes to diseases such as keratitis. An unmet need continues to exist for methods of preventing or treating a viral infection, including herpesviral infection, of a host.

### BRIEF SUMMARY OF THE INVENTION

The present invention provides, in one embodiment, a method of preventing or treating a viral infection of a host, the method comprising administering to the host an effective amount of an inhibitor of the EZH1 and/or EZH2 histone methyltransferase activities.

In another embodiment, the present invention provides a method of preventing or treating a viral infection of a host, the method comprising administering to the host an effective amount of a compound of Formula (I):

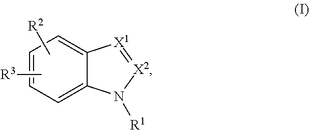

wherein X¹, X², R¹, R², and R³ are defined herein; or pharmaceutically acceptable salts, solvates, or stereoisomers thereof.

In another embodiment, the present invention provides a method of inhibiting an EZH1 or EZH2 methyltransferase in a virus-infected host, the method comprising administering to the host an effective amount of a compound of Formula (I) as defined above.

In another embodiment, the present invention provides a method of improving the therapeutic effect of a pharmaceutical composition, the method comprising adding to the pharmaceutical composition a compound of Formula (I) as defined above.

### BRIEF DESCRIPTION OF THE DRAWINGS

FIG. 1 is a line graph showing mRNA levels of herpes simplex virus 1 (HSV-1) viral immediate early (IE) genes and control genes in HSV-1 infected HFF (human foreskin fibroblast) cells treated with increasing concentrations of compound 1, in accordance with embodiments of the invention.

FIG. 2 is a line graph showing mRNA levels of HSV-1 viral IE genes and control genes in HSV-1 infected HFF cells treated with increasing concentrations of compound 2, in accordance with embodiments of the invention.

FIG. 3 is a line graph showing mRNA levels of HSV-1 viral IE genes and control genes in HIV-1 infected HFF cells treated with increasing concentrations of compound 3, in accordance with embodiments of the invention.

FIG. 4 is a bar graph showing mRNA levels of HSV-1 viral IE genes and control genes in HSV-1 infected HFF cells at increasing levels of HSV multiplicity of infection (MOI) and treated with compound 4, in accordance with embodiments of the invention.

FIG. 5 is a bar graph showing HSV-1 viral DNA levels in HSV-1 infected HFF cells isolated from total and nuclear cellular fractions treated with compound 1 or 4, in accordance with embodiments of the invention.

FIG. 6 is a line graph showing a time course of mRNA levels of HSV-1 viral IE genes and control genes in HSV-1 infected HFF cells treated with compound 4 at the indicated times relative to the time of infection (0 time), in accordance with embodiments of the invention.

FIG. 7 is a bar graph showing mRNA levels of human cytomegalovirus (hCMV) viral genes (UL37 and UL123 are IE genes; UL44 is an Early gene) and control genes in hCMV infected HFF cells treated with compound 1 or compound 4, in accordance with embodiments of the invention.

FIG. 8 is a bar graph showing mRNA levels of adenovirus 5 (ADV-5) viral genes (E1A is an IE gene) and control genes in ADV-5 infected HFF cells treated with compound 1 or compound 4, in accordance with embodiments of the invention.

FIG. 9 presents line graphs showing mRNA levels of HSV-1 viral IE genes and control genes in HSV-1 infected HFF cells treated with increasing concentrations of compounds 1 or 4, in accordance with embodiments of the invention.

FIG. 10 presents line graphs showing mRNA levels of HSV-1 viral IE genes and control genes in HSV-1 infected Vero cells treated with increasing concentrations of compound 1 or 4, in accordance with embodiments of the invention.

FIG. 11 is a bar graph showing mRNA levels of HSV-1 viral IE genes, control genes, and cellular innate interferon signaling genes in mock or HSV-1 infected HFF cells treated with compound 4, in accordance with embodiments of the invention.

FIG. 12 is a line graph showing a time course of mRNA levels of HSV-1 viral IE genes and control genes in HSV-1 infected HFF cells pretreated with compound 4 for various lengths of time prior to infection, in accordance with embodiments of the invention.

FIG. 13 presents dot plots of viral yield per trigeminal ganglia explanted from HSV-1 latently infected mice treated with ACV (Acyclovir) or ML324 (N-(3-(dimethylamino)propyl)-4-(8-hydroxyquinolin-6-yl)benzamide, in accordance with embodiments of the invention.

FIG. 14 is a dot plot of viral yield per trigeminal ganglia explanted from HSV-1 latently infected mice treated with compound 1 or compound 4, in accordance with embodiments of the invention.

FIG. 15 is a dot plot of viral yield per trigeminal ganglia explanted from HSV-1 latently infected mice treated with compound 3, in accordance with embodiments of the invention.

FIG. 16 is a dot plot of viral DNA yield per trigeminal ganglia explanted from HSV-1 latently infected mice treated with compound 1 or compound 4, in accordance with embodiments of the invention.

FIG. 17 is a dot plot of viral DNA yield per trigeminal ganglia explanted from HSV-1 latently infected mice treated with ACV and ML324, in accordance with embodiments of the invention.

FIG. 18 is a dot plot of the total number of UL29+ individual/single neurons per trigeminal ganglia explanted from HSV-1 latently infected mice treated with ACV, ML324, compound 1, and compound 4. This reflects the number of neurons in which HSV is undergoing reactivation in a given ganglia, in accordance with embodiments of the invention.

FIG. 19 is a dot plot of UL29+ neuron clusters per trigeminal ganglia explanted from HSV-1 latently infected mice treated with ACV, ML324, compound 1, and compound 4. This reflects the number of primary reactivating neurons with spread to surrounding cells and is a measure of the inhibition of transmission rather than initiating reactivation events, in accordance with embodiments of the invention.

FIG. 20 is a volcano plot showing the results (n=3) of microarray analysis on HFF cells after treatment with an EZH1/2 inhibitor, in accordance with embodiments of the invention. The dotted line indicates a cutoff p-value of 0.05; above the dotted line is less than 0.05 p-value (negative log₁₀).

FIG. 21 is a line graph showing removal of compound 1 prior to infection leads to the recovery of HSV IE expression and a decrease in IL6 stimulation. Levels of cellular innate signaling (IL-6), control (SP1, TBP), and HSV viral IE (ICP4, ICP22, ICP27) mRNAs are expressed relative to cells treated with DMSO (vehicle).

FIG. 22 is a line graph showing removal of compound 2 prior to infection leads to the recovery of HSV IE expression and a decrease in IL6 stimulation. Levels of cellular innate signaling (IL-6), control (SP1, TBP), and HSV viral IE (ICP4, ICP22, ICP27) mRNAs are expressed relative to cells treated with DMSO (vehicle).

FIG. 23 is a line graph showing removal of compound 3 prior to infection leads to the recovery of HSV IE expression. Levels of cellular innate signaling (IL-6), control (SP1, TBP), and HSV viral IE (ICP4, ICP22, ICP27) mRNAs are expressed relative to cells treated with DMSO (vehicle).

FIG. 24 is a line graph showing removal of compound 4 prior to infection leads to the recovery of HSV IE expression and a decrease in IL6 stimulation. Levels of cellular innate signaling (IL-6), control (SP1, TBP), and HSV viral IE (ICP4, ICP22, ICP27) mRNAs are expressed relative to cells treated with DMSO (vehicle).

FIG. 25 presents dot plots showing suppression of EZH1/2 catalytic activity reduces HSV reactivation, in sensory neurons, and spread, within the sensory ganglia.

FIG. 26 is a line graph showing an EZH1/2 inhibitor induces the expression of innate gene expression in explanted ganglia. Levels of cellular innate signaling (IL-6, IL1b) and control (SP1, TBP) mRNAs are expressed relative to ganglia treated with DMSO (vehicle).

FIG. 27 presents dot plots showing viral DNA yield per eye and per ganglia determined through quantitative real-time PCR.

FIG. 28 is presents dot plots showing viral yield (pfu) determined by titering on Vero cells.

FIG. 29 shows Western blot of IE proteins (ICP4, ICP27) and the ratios to levels in DMSO treated cells, normalized to the actin-loading control.

FIG. 30 is a line graph that shows an EZH1/2 inhibitor suppresses lytic HSV gene expression in MRC-5 fibroblast cells.

FIG. 31 is a bar graph that shows EZH1/2 inhibitors block the spread of lytic HSV infection.

FIG. 32 presents line graphs that show the number and size of Focus Forming Units (FFU) after HFF cells were treated with the indicated concentrations of compound 4 for 5 h and infected with ZIKV for 40 h, in accordance with embodiments of the invention.

FIG. 33 is a line graph that shows the percent of cells infected at days 1 and 2 treated with the indicated concentrations of compound 4, in accordance with embodiments of the invention.

FIG. 34 is a line graph that shows the results after cells were treated either preadsorption or post-adsorption when the percent of cells infected was determined at days 1 and 2, in accordance with embodiments of the invention.

### DETAILED DESCRIPTION OF THE INVENTION

The modulation of chromatin associated with the herpes simplex virus (HSV) genome regulates both viral lytic replication and the latency-reactivation cycles. This dynamic process is based on the interplay of epigenetic machinery that can result in either heterochromatic suppression or euchromatic activation of viral gene expression. Inhibition of key epigenetic factors that promote the euchromatic state of the viral genome can shift the chromatin dynamic, resulting in suppression of lytic infection and a block to viral reactivation from latency. Additionally, this dynamic can be modulated by cellular antiviral pathways (i.e. innate immunity).

In contrast to chromatin modulators that promote viral gene expression, the histone methyltransferase host proteins Enhancer of Zeste Homologs 1 and 2 (EZH1 and EZH2) have been implicated in the suppression of infection and in the maintenance of viral latency via installation of repressive historic H3-lysine 27-methylation in chromatin associated with the viral genome. Surprisingly and unexpectedly, however, compounds that inhibit the catalytic activity of these repressors, or block their interactions with cofactors of the repressive complex with which they are associated, result in repression rather than the anticipated activation of viral (IE) gene expression. Additionally, inhibition of EZH1/2 also suppresses the initiation of viral reactivation from latency as shown by a reduction in the number of primary neurons undergoing viral reactivation in the mouse ganglia explant model.

The exact role of EZH1 versus EZH2 in various contexts is not known. One line of research suggests that there is a catalytic specificity distinction (H3K27-me1 versus me2/3), while others suggest that the two proteins are differentially expressed during cell cycle or development. Finally, a third suggests that EZH1, in certain cases, is an activator via installation of H3K27-me1 while EZH2 is primarily a repressor via installation of H3K27-me2/3). Without wishing to be bound to any theory or mechanism, the inhibitors block the initiation stage of HSV infection and reactivation. This is distinct from the later stage of infection/reactivation, which can be suppressed by DNA replication inhibitors (e.g., acyclovir and derivatives). Inhibition of the initiation stage of infection/reactivation prevents viral shedding, inflammation contributing to keratitis or transplant rejection, and transmission during childbirth.

The present invention provides, in one embodiment, a method of preventing or treating a viral infection of a host, the method comprising administering to the host an effective amount of an inhibitor of the EZH1 and/or EZH2 histone methyltransferase activities.

The present invention provides, in one embodiment, a method of preventing or treating a viral infection of a host, the method comprising administering to the host an effective amount of a compound of Formula (I):

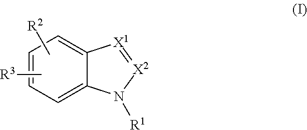

wherein X¹ and X² are each CR⁴, X¹ is N and X² is CR⁴, or X¹ is CR⁴ and X² is N; R¹ is alkyl optionally substituted with one or more substituents selected from cycloalkyl, heterocycloalkyl, aryl, and heteroaryl, each substituent optionally further substituted with one or more substituents selected from halo, alkyl, amino, nitro, cyano, and alkoxyl; R²is H or —L—NR⁵—(CH₂)ₘ—X³, is SO₂ or CO, m is 0 to 3, X³ is H, cycloalkyl, heterocycloalkyl, aryl, or heteroaryl, each cycloalkyl, heterocycloalkyl, aryl, and heteroaryl optionally substituted with one or more substituents selected from halo, alkyl, amino, nitro, cyano, and alkoxyl, the cycloalkyl and heterocycloalkyl optionally having an unsubstituted methylene group replaced by CO; R³ is H, cycloalkyl, heterocycloalkyl, aryl, or heteroaryl, each cycloalkyl, heterocycloalkyl, aryl, and heteroaryl optionally substituted with one or more substituents selected from halo, alkyl, amino, nitro, cyano, alkoxyl, cycloalkyl, heterocycloalkyl, aryl, and heteroaryl, each optional substituent cycloalkyl, heterocycloalkyl, aryl, and heteroaryl optionally further substituted with one or more substituents selected from alkyl, amino, nitro, cyano, and alkoxyl; R⁴ is H, alkyl, or NR⁶R⁷; R⁵ is H or alkyl; R⁶ is H, cycloalkyl, heterocycloalkyl, aryl, or heteroaryl, each cycloalkyl, heterocycloalkyl, aryl, and heteroaryl optionally substituted with one or more substituents selected from halo, alkyl, amino, nitro, cyano, alkoxyl, cycloalkyl, heterocycloalkyl, aryl, and heteroaryl, each optional substituent alkyl, cycloalkyl, heterocycloalkyl, aryl, and heteroaryl optionally further substituted with one or more substituents selected from alkyl, amino, nitro, cyano, alkoxyl, cycloalkyl, heterocycloalkyl, aryl, and heteroaryl, each further optional substituent cycloalkyl, heterocycloalkyl, aryl, and heteroaryl optionally substituted with one or more substituents selected from alkyl, amino, nitro, cyano, and alkoxyl; and R⁷ is H or alkyl; or a pharmaceutically acceptable salt, solvate, or stereoisomer thereof.

In another embodiment, the present invention provides a method of inhibiting an EZH1 or EZH2 methyltransferase in a virus-infected host, the method comprising administering to the host an effective amount of a compound of Formula (I) as defined above.

In another embodiment, R¹ is C₁-C₄ alkyl optionally substituted with phenyl, the phenyl optionally further substituted with fluorine. In another embodiment, R¹ is isopropyl, 4-fluorobenzyl, or 2-butyl.

In another embodiment, R² is —L—NR⁵—(CH₂)ₘ—X³. In another embodiment, L is CO. In another embodiment, R² is —CO—NH—(CH₂)-heterocycloalkyl, the heterocycloalkyl optionally substituted with alkyl and optionally having an unsubstituted methylene group replaced by CO. In another embodiment, R² is

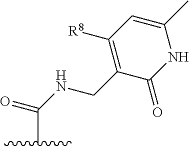

wherein R⁸ is methyl or n-propyl.

In another embodiment, R³ is heteroaryl optionally substituted with heterocycloalkyl, the heterocycloalkyl optionally further substituted with alkyl. In another embodiment, R³ pyridinyl substituted with piperazinyl, the piperazinyl optionally further substituted with alkyl. In another embodiment, R³ is

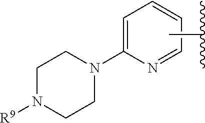

wherein R⁹ is H, methyl, or isopropyl.

In another embodiment, R⁴ is methyl or NH-(heterocycloalkyl), the heterocycloalkyl optionally substituted with alkyl, the alkyl optionally further substituted with aryl, the aryl optionally further substituted with alkoxyl. In another embodiment, R⁴ is NH-(piperidinyl)-(alkyl)-(phenyl)-alkoxyl. In another embodiment, R⁴ is

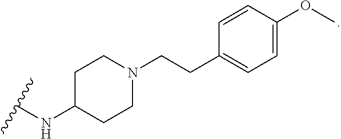 [SMILES: *NC1CCN(CCc2ccc(OC)cc2)CC1]

In another embodiment, R¹ is isopropyl, 4-fluorobenzyl, or 2-butyl; R² is

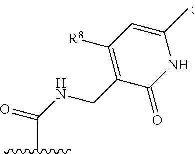

### R³ is

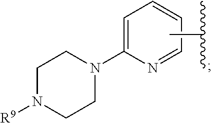

### R⁴ is

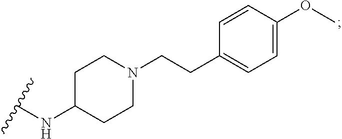 [SMILES: *NC1CCN(CCc2ccc(OC)cc2)CC1]

R⁸ is methyl or n-propyl; and R⁹ is H, methyl, or isopropyl.

In another embodiment, the compound is a compound of Formula (II):

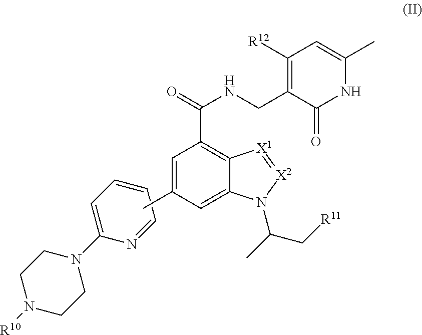

wherein X¹ and X² are each CR⁴ or X¹ is CR⁴ and X² is N; R⁴ is H or methyl; R¹⁰ is H, methyl, ethyl, or propyl; R¹¹ is H or methyl; and R¹² is methyl, ethyl, or propyl.

In another embodiment, the compound is

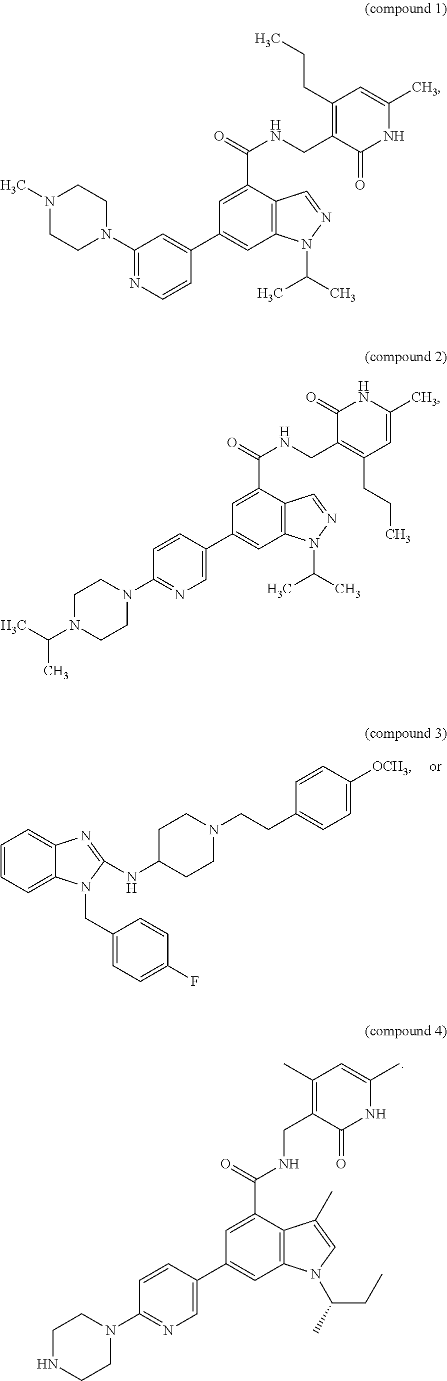 [SMILES: CCCc1cc(C)[nH]c(=O)c1CNC(=O)c1cc(-c2ccnc(N3CCN(C)CC3)c2)cc2c1cnn2C(C)C ; CCCc1cc(C)[nH]c(=O)c1CNC(=O)c1cc(-c2ccc(N3CCN(C(C)C)CC3)nc2)cc2c1cnn2C(C)C ; COc1ccc(CCN2CCC(Nc3nc4ccccc4n3Cc3ccc(F)cc3)CC2)cc1 ; CC[C@H](C)n1cc(C)c2c(C(=O)NCc3c(C)cc(C)[nH]c3=O)cc(-c3ccc(N4CCNCC4)nc3)cc21]

Compound 1 is known as GSK343, compound 2 is known as UNC1999, compound 3 is known as astemizole, and compound 4 is known as GSK126. All are commercially available. For example, compounds 1-3 are available, e.g. from Sigma-Aldrich (St. Louis, Mo., USA), catalog nos. SML0766, SML0778, and A2861, respectively. Compound 4 is available, e.g., from EMD Millipore (Billerica, Mass., USA), catalog no. 500580.

In any of the embodiments above, the teen “alkyl” implies a straight-chain or branched alkyl containing, for example, from 1 to 6 carbon atoms, e.g., from 1 to 4 carbon atoms. Examples of alkyl group include methyl, ethyl, n-propyl, isopropyl, n-butyl, sec-butyl, isobutyl, tert-butyl, n-pentyl, isopentyl, n-hexyl, and the like. This definition also applies wherever “alkyl” occurs as part of a group, such as, e.g., fluoro C₁-C₆ alkyl. The alkyl may be substituted or unsubstituted, as described herein.

In any of the embodiments above, the term “cycloalkyl,” as used herein, means a cyclic alkyl moiety containing from, for example, 3 to 6 carbon atoms or from 5 to 6 carbon atoms. Examples of such moieties include cyclopropyl, cyclobutyl, cyclopentyl, cyclohexyl, and the like. The cycloalkyl may be substituted or unsubstituted, as described herein.

The term “heterocycloalkyl,” as used herein, means a stable, saturated, or partially unsaturated monocyclic, bicyclic, and spiro ring system containing 3 to 7 ring members of carbon atoms and other atoms selected from nitrogen, sulfur, and/or oxygen, the ring system containing optionally one of two double bonds. In an aspect, a heterocycloalkyl is a 5, 6, or 7-membered monocyclic ring and contains one, two, or three heteroatoms selected from nitrogen, oxygen, and sulfur. The heterocycloalkyl may be attached to the parent structure through a carbon atom or through any heteroatom of the heterocycloalkyl that results in a stable structure. Examples of such heterocycloalkyl rings are isoxazolyl, thiazolinyl, imidazolidinyl, piperazinyl, homopiperazinyl, pyrrolinyl, pyrrolidinyl, pyrazolyl, pyranyl, piperidyl, oxazolyl, and morpholinyl. The heterocycloalkyl may be substituted or unsubstituted, as described herein.

In any of the embodiments above, the term “hydroxyl” refers to the group —OH.

In any of the embodiments above, the terms “alkoxyl” and “aryloxyl” refer to linear or branched alkyl and aryl groups that are attached to a divalent oxygen. The alkyl and aryl groups are the same as described herein.

In any of the embodiments above, the term “halo” refers to a halogen selected from fluorine, chlorine, bromine, and iodine.

In any of the embodiments above, the term “aryl” refers to a mono, bi, or tricyclic carbocyclic ring system that may have one, two, or three aromatic rings, for example, phenyl, naphthyl, anthracenyl, or biphenyl. The term “aryl” refers to an unsubstituted or substituted aromatic carbocyclic moiety, as commonly understood in the art, and includes monocyclic and polycyclic aromatics such as, for example, phenyl, biphenyl, naphthyl, anthracenyl, pyrenyl, and the like. An aryl moiety generally contains from, for example, 6 to 30 carbon atoms, from 6 to 18 carbon atoms, from 6 to 14 carbon atoms, or from 6 to 10 carbon atoms. It is understood that the term aryl includes carbocyclic moieties that are planar and comprise 4n+2π electrons, according to Hückel's Rule, wherein n=1, 2, or 3. The aryl may be substituted or unsubstituted as described herein.

In any of the embodiments above, the term “heteroaryl” refers to an aryl as defined above in which at least one, preferably 1 or 2, of the carbon atoms of the aromatic carbocyclic ring is replaced by N, O or S atoms. Examples of heteroaryl include pyridyl, furanyl, pyrrolyl, quinolinyl, thiophenyl, indolyl, imidazolyl and the like.

In other aspects, any substituent that is not hydrogen (e.g., C₁-C₆ alkyl, C₂-C₆ alkenyl, C₃-C₆ cycloalkyl or aryl) may be an optionally substituted moiety. The substituted moiety typically comprises at least one substituent (e.g., 1, 2, 3, 4, 5, 6, etc.) in any suitable position (e.g., 1-, 2-, 3-, 4-, 5-, or 6-position, etc.). When an aryl group is substituted with a substituent, e.g., halo, amino, alkyl, OH, alkoxy, cyano, nitro, and others, the aromatic ring hydrogen is replaced with the substituent and this may take place in any of the available hydrogens, e.g., 2, 3, 4, 5, and/or 6-position wherein the 1-position is the point of attachment of the aryl group in the compounds, salts, solvates, or stereoisomers of the present invention. Suitable substituents include, e.g., halo, alkyl, alkenyl, alkynyl, hydroxy, nitro, cyano, amino, alkylamino, alkoxy, aryloxy, aralkoxy, carboxyl, carboxyalkyl, carboxyalkyloxy, amido, alkylamido, haloalkylamido, aryl, heteroaryl, and heterocycloalkyl. In some instances, the substituent is at least one alkyl, halo, and/or haloalkyl (e.g., 1 or 2).

In any of the embodiments above, whenever a range of the number of atoms in a structure is indicated (e.g., a C₁-C₆, or C₁-C₄ alkyl, C₃-C₆ cycloalkyl, etc.), it is specifically contemplated that any sub-range or individual number of carbon atoms falling within the indicated range also may be used. Thus, for instance, the recitation of a range of 1-6 carbon atoms (e.g., C₁-C₆), 1-4 carbon atoms (e.g., C₁-C₄), 1-3 carbon atoms (e.g., C₁-C₃), or 2-6 carbon atoms (e.g., C₂-C₆) as used with respect to any chemical group (e.g., alkyl, cycloalkyl, etc.) referenced herein encompasses and specifically describes 1, 2, 3, 4, 5, and/or 6 carbon atoms, as appropriate, as well as any sub-range thereof (e.g., 1-2 carbon atoms, 1-3 carbon atoms, 1-4 carbon atoms, 1-5 carbon atoms, 1-6 carbon atoms, 2-3 carbon atoms, 2-4 carbon atoms, 2-5 carbon atoms, 2-6 carbon atoms, 3-4 carbon atoms, 3-5 carbon atoms, 3-6 carbon atoms, 4-5 carbon atoms, 4-6 carbon atoms, etc., as appropriate).

A salt of a compound is a biologically acceptable salt, which is generally non-toxic, and is exemplified by salts with base or acid addition salts, inclusive of salts with inorganic base such as alkali metal salt (e.g., a sodium salt, a potassium salt), alkaline earth metal salt (e.g., calcium salt, magnesium salt), ammonium salt, salts with organic base such as organic amine salt (e.g., triethylamine salt, diisopropylethylamine salt, pyridine salt, picoline salt, ethanolamine salt, diethanolamine salt, triethanolamine salt, dicyclohexylamine salt, N,N′-dibenzylethylenediamine salt), inorganic acid salt (e.g., hydrochloride, hydrobromide, sulfate, phosphate), organic carboxylic or sulfonic acid salt (e.g., formate, acetate, trifluoroacetate maleate, tartrate, fumarate, methanesulfonate, benzenesulfonate, toluenesulfonate), salt with basic or acid amino acid (e.g., arginine, aspartic acid, glutamic acid), and the like. In any of the embodiments above, the term “salt” encompasses “pharmaceutically acceptable salt.” Lists of suitable pharmaceutical salts are found in, for example, Remington's Pharmaceutical Sciences, 18th ed., Mack Publishing Company, Easton, Pa., 1990, p. 1445, and Journal of Pharmaceutical Science, 66, 2-19 (1977). For example, they may be a salt of an alkali metal (e.g., sodium or potassium), alkaline earth metal (e.g., calcium), or ammonium of salt.

Salts formed from free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

It is further understood that the compounds described herein may form solvates, or exist in a substantially uncomplexed form, such as the anhydrous form. Those of skill in the art appreciate that many organic compounds can form complexes with solvents in which they are reacted or from which they are precipitated or crystallized. These complexes are known as “solvates.” A solvate is a molecule consisting of a complex made up of solute molecules and solvent molecules resulting from the solution. For example, a complex with water is known as a “hydrate.” Solvates as defined herein may be crystalline or non-crystalline, such as amorphous, and may be formed by any suitable method, including, but not limited to reaction, precipitation, or crystallization. Solvates of the compounds, salts, and stereoisomers described herein, including pharmaceutically acceptable solvates, are within the scope of the invention.

It will also be appreciated by those of skill in the art that many organic compounds can exist in more than one crystalline form (polymorphic forms). For example, crystalline form may vary from solvate to solvate. Thus, all crystalline forms of the compounds, salts, solvates, and stereoisomers described herein are within the scope of the present invention. Pharmaceutically acceptable solvates include hydrates, alcoholates such as methanolates and ethanolates, acetonitrilates and the like.

A compound can have stereoisomers based on asymmetric carbon atoms and double bonds, such as optical isomers, geometric isomers, and the like, all of which and mixtures thereof are also encompassed in the present invention.

The compounds, salts, solvates, or stereoisomers of Formula (I) may be prepared by any suitable synthetic methodology.

The methods described herein comprise administering a compound, salt, solvate, or stereoisomer of Formula (I) in the form of a composition, e.g., a pharmaceutically acceptable composition. In particular, a composition will comprise at least one compound, salt, solvate, or stereoisomer of Formula (I) and a pharmaceutically acceptable carrier. The pharmaceutically acceptable excipients described herein, for example, vehicles, adjuvants, carriers or diluents, are well-known to those who are skilled in the art and are readily available to the public. Typically, the pharmaceutically acceptable carrier is one that is chemically inert to the active compound, salt, solvate, or stereoisomer and one that has no detrimental side effects or toxicity under the conditions of use.

The compositions may be administered as oral, sublingual, transdermal, subcutaneous, topical, absorption through epithelial or mucocutaneous linings, intravenous, intranasal, intraarterial, intramuscular, intratumoral, peritumoral, interperitoneal, intrathecal, rectal, vaginal, or aerosol formulations. In some aspects, the composition is administered orally or intravenously.

In accordance with any of the embodiments, a compound, salt, solvate, or stereoisomer of Formula (I) may be administered orally to a subject in need thereof. Formulations suitable for oral administration may consist of (a) liquid solutions, such as an effective amount of the compound, salt, solvate, or stereoisomer dissolved in diluents, such as water, saline, or orange juice and include an additive, such as cyclodextrin (e.g., α-, β-, or γ-cyclodextrin, hydroxypropyl cyclodextrin) or polyethylene glycol (e.g., PEG400); (b) capsules, sachets, tablets, lozenges, and troches, each containing a predetermined amount of the active ingredient, as solids or granules; (c) powders; (d) suspensions in an appropriate liquid; and (e) suitable emulsions and gels. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms may be of the ordinary hard- or soft-shelled gelatin type containing, for example, surfactants, lubricants, and inert fillers, such as lactose, sucrose, calcium phosphate, and cornstarch. Tablet forms may include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms may comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art.

Formulations suitable for parenteral administration include aqueous and non-aqueous, isotonic sterile injection solutions, which may contain anti-oxidants, buffers, bacteriostats, and solutes that render the formulation isotonic with the blood of the intended recipient, and aqueous and non-aqueous sterile suspensions that may include suspending agents, solubilizers, thickening agents, stabilizers, and preservatives. The compound, salt, solvate, or stereoisomer of Formula (I) may be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which may be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl amnionium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene-polypropylene copolymers, (d) amphoteric detergents such as, for example, alkyl-beta-aminopropionates, and 2-alkyl-imidazoline quaternary ammonium salts, and (e) mixtures thereof.

The parenteral formulations will typically contain from about 0.5 to about 25% by weight of the compound, salt, solvate, or stereoisomer of Formula (I) in solution. Suitable preservatives and buffers may be used in such formulations. In order to minimize or eliminate irritation at the site of injection, such con positions may contain one or more nonionic surfactants having a hydrophile-lipophile balance (HLB) of from about 12 to about 17. The quantity of surfactant in such formulations ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations may be presented in unit-dose or multi-dose sealed containers, such as ampoules and vials, and may be stored in a freeze-dried (lyophilized) condition requiring only the addition of the sterile liquid carrier, for example, water, for injections, immediately prior to use. Extemporaneous injection solutions and suspensions may be prepared from sterile powders, granules, and tablets of the kind previously described.

The compound, salt, solvate, or stereoisomer of Formula (I) may be made into an injectable formulation. The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986).

Topically applied compositions are generally in the form of liquids (e.g., mouthwash), creams, pastes, lotions and gels. Topical administration includes application to any region of the skin. Topical administration also includes application to the oral mucosa, which includes the oral cavity, oral epithelium, palate, gingival, and the nasal mucosa. Topical administration also includes application to the eye, for example, using eye drops. Topical administration also includes application to the vagina, for example, as a vaginal gel or wash. In some embodiments, the composition contains at least one active component and a suitable vehicle or carrier. It may also contain other components, such as an anti-irritant. The carrier may be a liquid, solid or semi-solid. In embodiments, the composition is an aqueous solution, such as a mouthwash. Alternatively, the composition may be a dispersion, emulsion, gel, lotion or cream vehicle for the various components. In one embodiment, the primary vehicle is water or a biocompatible solvent that is substantially neutral or that has been rendered substantially neutral. The liquid vehicle may include other materials, such as buffers, alcohols, glycerin, and mineral oils with various emulsifiers or dispersing agents as known in the art to obtain the desired pH, consistency and viscosity. It is possible that the compositions may be produced as solids, such as powders or granules. The solids may be applied directly or dissolved in water or a biocompatible solvent prior to use to form a solution that is substantially neutral or that has been rendered substantially neutral and that may then be applied to the target site. In embodiments of the invention, the vehicle for topical application to the skin may include water, buffered solutions, various alcohols, glycols such as glycerin, lipid materials such as fatty acids, mineral oils, phosphoglycerides, collagen, gelatin and silicone based materials.

The compound, salt, solvate, or stereoisomer of Formula (I), alone or in combination with other suitable components, may be made into aerosol formulations to be administered via inhalation. These aerosol formulations may be placed into pressurized acceptable propellants, such as dichlorodifluoromethane, propane, nitrogen, and the like. They also may be formulated as pharmaceuticals for non-pressured preparations, such as in a nebulizer or an atomizer.

It will be appreciated by one of ordinary skill in the art that, in addition to the aforedescribed compositions, a compound, salt, solvate, or stereoisomer of the invention may be formulated as inclusion complexes, such as cyclodextrin inclusion complexes, or liposomes. Liposomes may serve to target a compound, salt, solvate, or stereoisomer of the invention to a particular tissue, such as lymphoid tissue or cancerous hepatic cells. Liposomes may also be used to increase the half-life of a compound, salt, solvate, or stereoisomer of the invention. Many methods are available for preparing liposomes, as described in, for example, Szoka et al., Ann. Rev. Biophys. Bioeng. 1980, 9, 467 and U.S. Pat. Nos. 4,235,871, 4,501,728, 4,837,028, and 5,019,369.

A “host” may be considered a single cell, a tissue, an organ, or an individual organism, such as a mammal. The mammal may be any suitable mammal, such as a mammal selected from the group consisting of a mouse, rat, guinea pig, hamster, cat, dog, pig, cow, horse, and primate. In one embodiment, the mammal is a human.

In one embodiment, the viral infection involves reactivation of a virus after latency in the host. In another embodiment, the viral infection is due to a herpesvirus or adenovirus or flavivirus.

A viral infection is present in a host when a virus replicates itself within the host. A virus contains its own genetic material but uses the machinery of the host to reproduce. The virus may reproduce immediately, whereby the resulting virions destroy a host cell to attack additional cells. This process is the viral lytic cycle. Alternatively, a virus may establish a quiescent infection in a host cell, lying dormant until environmental stimuli trigger re-entry into the lytic replication cycle. Such re-emergence or re-entry into the lytic replication cycle is termed reactivation. In an embodiment of the invention, the host has a viral infection or is at risk for viral infection but is free from cancer. In some embodiments of the invention, the viral infection may be any of chronic, severe, and/or acute with clinical symptoms or may be subclinical viral shedding. EZH1/2 inhibitors may be used as anti-parasitic/anti-microbial therapies as well.

The viral infection may be due to a nuclear DNA viral infection such as a herpes viral infection. The herpesvirus may be, e.g., herpes simplex virus type 1 (HSV-1, HHV-1), herpes simplex virus type 2 (HSV-2, HHV-2), varicella zoster virus (VZV, HHV-3), or cytomegalovirus (CMV, HHV-5). The herpesvirus may be Epstein-Barr virus (EBV, HHV-4), Kaposi's Sarcoma-Associated herpesvirus (HHV-8), human herpesvirus-6A/B or human herpesvirus-7. The virus may be adenovirus (ADV), e.g., ADV type 5.

The viral infection may be due to an RNA virus. An example of an RNA virus includes flaviviruses, e.g., the Zika virus.

Viral infections especially pose a threat to individuals that have suppressed (immunosuppressed) or otherwise compromised (immunocompromised) immune systems. For example, individuals with HIV/AIDS, diabetes, or cancer often have reduced ability to ward off additional and/or opportunistic viral infections due to immune systems that are adversely affected by the underlying, primary infection or condition. Therefore, preventing or treating viral infection or re-activation is especially important for these individuals.

Another embodiment of the invention provides a method of preventing or treating a viral infection in a mammal that has undergone, is undergoing, or will undergo an organ or tissue transplant, comprising administering to the mammal an effective amount of any of the compounds described above, wherein the administration of the inhibitor(s) prevents or treats the viral infection. A non-limiting example would be to administer an effective amount of an inhibitor of EZH1 or EZH2 to a mammal undergoing immunosuppressive therapy and who is suspected of being infected with virus.

Other inhibitors of EZH1 or EZH2 may be used alone or in combination. A suitable inhibitor includes a nucleic acid (e.g., siRNA, sbRNA), protein, small molecule, or antibody that specifically binds to a EZH1 or EZH2, inhibits translation of EZH1 or EZH2, inhibits transcription of EZH1 or EZH2, or otherwise interferes with the biological expression and/or activity of EZH1 or EZH2. One such inhibitors an RNA interference (RNAi) inhibitor. The RNAi inhibitor may comprise any RNA sequence that is complementary to the target EZH1 or EZH2 nucleic acid or a portion thereof, and include small inhibitor RNA (siRNA). Antibodies and RNAi inhibitors of EZH1 or EZH2 may be prepared using routine techniques.

The terms “treat,” “prevent,” and “inhibit” as weld as words stemming therefrom, as used herein, do not necessarily imply 100% or complete treatment, prevention, or inhibition. Rather, there are varying degrees of treatment, prevention, or inhibition of which one of ordinary skill in the art recognizes as having a potential benefit or therapeutic effect. In this respect, the inventive methods may provide any amount of any level of treatment, prevention, or inhibition of a condition associated with, e.g., EZH1 or EZH2 activity, such as methylation of histones, in a host. Furthermore, the treatment, prevention, or inhibition provided by the inventive methods may include treatment, prevention, or inhibition of one or more conditions or symptoms of the disease being treated, prevented, or inhibited. Also, for purposes herein, “prevention” or “inhibiting” may encompass delaying the onset of the disease or a symptom or condition thereof.

An “effective amount” refers to a dose that is adequate to prevent, treat, or inhibit a condition associated with, e.g., EZH1 or EZH2 histone transmethylase activity. Amounts effective for a therapeutic or prophylactic use will depend on, for example, the stage and severity of the disease or disorder being treated, the age, weight, and general state of health of the patient, and the judgment of the prescribing physician. The size of the dose will also be determined by the compound selected, method of administration, timing and frequency of administration as well as the existence, nature, and extent of any adverse side-effects that might accompany the administration of a particular compound and the desired physiological effect. For example, the dose of the inhibitor to be administered for treating a condition associated with, e.g., EZH1 or EZH2 histone transmethylase activity, may be about 0.1 mg to about 10 g per day (e.g., 0.5 mg, 1 mg, 5 mg, 10 mg, 20 mg, 30 mg, 40 mg, 50 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 2 g, 3 g, 4 g, 5 g, 6 g, 7 g, 8 g, 9 g, or ranges of any of the values described herein). The dose of the inhibitor to be administered for preventing a condition associated with, e.g., EZH1 or EZH2 histone transmethylase activity, may be less than the dose for treating such a condition, e.g. about 0.001 mg/kg per day to about 1 mg/kg per day (e.g., 0.001 mg/kg, 0.005 mg/kg, 0.01 mg/kg, 0.05 mg/kg, 0.1 mg/kg, 0.5 mg/kg, 1 mg/kg, or ranges of any of the values described herein). Alternatively or in addition, the dose of inhibitor to be administered for prevention or treatment may be 0.001 mg/kg to 200 mg/kg per day (e.g., 0.01 mg/kg, 0.05 mg/kg, 0.1 mg/kg, 0.5 mg/kg, 1 mg/kg, 5 mg/kg, 10 mg/kg, 50 mg/kg, 100 mg/kg, 150 mg/kg, or ranges of any of the values described herein). It will be appreciated by one of skill in the art that various diseases or disorders could require prolonged treatment involving multiple administrations, e.g., using inhibitors of EZH1 or EZH2 in each or various rounds of administration.

A compound, salt, solvate, or stereoisomer of Formula (I) may be administered, simultaneously or sequentially or cyclically, in a coordinate protocol with one or more secondary or adjunctive agents. Thus, in certain embodiments compound, salt, solvate, or stereoisomer of Formula (I) is administered coordinately with a different agent, or any other secondary or adjunctive agent, utilizing separate formulations or a combinatorial formulation as described above (i.e., comprising both compound, salt, solvate, or stereoisomer of Formula (I) and another agent). This coordinate administration may be done simultaneously or sequentially in either order, and there may be a time period while only one or both (or all) active agents individually and/or collectively exert their biological activities. In another embodiment, the EZH1/2 inhibitors described herein may themselves be used as adjuvants since they induce pro-inflammatory cytokines, chemokines, and adhesion proteins involved in innate signaling and the recruitment of immune infiltrating cells (neutrophils) involved in both viral clearance and inflammation. Thus, in an embodiment, the present invention provides a method of improving the therapeutic effect of a pharmaceutical composition, the method comprising adding to the pharmaceutical composition a compound of Formula (I) as defined herein.

The following includes certain aspects of the invention.

1. A method of preventing or treating a viral infection of a host, the method comprising administering to the host an effective amount of an inhibitor of the EZH1 and/or EZH2 historic methyltransferase activities.

2. The method of aspect 1, wherein the inhibitor is a compound of Formula (I):

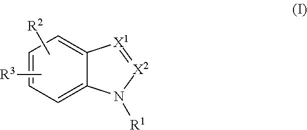

wherein
X¹ and X² are each CR⁴, X¹ is N and X² is CR⁴, or X¹ is CR⁴ and X² is N;
R¹ is alkyl optionally substituted with one or more substituents selected from cycloalkyl, heterocycloalkyl, aryl, and heteroaryl, each substituent optionally further substituted with one or more substituents selected from halo, alkyl, amino, nitro, cyano, and alkoxyl;
R² is H or —L—NR⁵—(CH₂)ₘ—X³,

### L is SO₂ or CO,

m is 0 to 3,
X³ is H, cycloalkyl, heterocycloalkyl, aryl, or heteroaryl, each cycloalkyl, heterocycloalkyl, aryl, and heteroaryl optionally substituted with one or more substituents selected from halo, alkyl, amino, nitro, cyano, and alkoxyl, the cycloalkyl and heterocycloalkyl optionally having an unsubstituted methylene group replaced by CO;
R³ is H, cycloalkyl, heterocycloalkyl, aryl, or heteroaryl, each cycloalkyl, heterocycloalkyl, aryl, and heteroaryl optionally substituted with one or more substituents selected from halo, alkyl, amino, nitro, cyano, alkoxyl, cycloalkyl, heterocycloalkyl, aryl, and heteroaryl, each optional substituent cycloalkyl, heterocycloalkyl, aryl, and heteroaryl optionally further substituted with one or more substituents selected from alkyl, amino, nitro, cyano, and alkoxyl;
R⁴ is H, alkyl, or NR⁶R⁷;
R⁵ is H or alkyl;
R⁶ is H, cycloalkyl, heterocycloalkyl, aryl, or heteroaryl, each cycloalkyl, heterocycloalkyl, aryl, and heteroaryl optionally substituted with one or more substituents selected from halo, alkyl, amino, nitro, cyano, alkoxyl, cycloalkyl, heterocycloalkyl, aryl, and heteroaryl, each optional substituent alkyl, cycloalkyl, heterocycloalkyl, aryl, and heteroaryl optionally further substituted with one or more substituents selected from alkyl, amino, nitro, cyano, alkoxyl, cycloalkyl, heterocycloalkyl, aryl, and heteroaryl, each further optional substituent cycloalkyl, heterocycloalkyl, aryl, and heteroaryl optionally substituted with one or more substituents selected from alkyl, amino, nitro, cyano, and alkoxyl; and
R⁷ is H or alkyl;
or a pharmaceutically acceptable salt, solvate, or stereoisomer thereof.

3. A method of inhibiting an EZH1 or EZH2 methyltransferase in a virus-infected host, the method comprising administering to the host an effective amount of a compound of Formula (I):

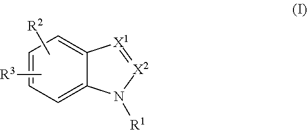

wherein
X¹ and X² are each CR⁴, X¹ is N and X² is CR⁴, or X¹ is CR⁴ and X² is N;
R¹ is alkyl optionally substituted with one or more substituents selected from cycloalkyl, heterocycloalkyl, aryl, and heteroaryl, each substituent optionally further substituted with one or more substituents selected from halo, alkyl, amino, nitro, cyano, and alkoxyl;
R² is H or —L—NR⁵—(CH₂)ₘ—X³,

### L is SO₂ or CO,

m is 0 to 3,
X³ is H, cycloalkyl, heterocycloalkyl, aryl, or heteroaryl, each cycloalkyl, heterocycloalkyl, aryl, and heteroaryl optionally substituted with one or more substituents selected from halo, alkyl, amino, nitro, cyano, and alkoxyl, the cycloalkyl and heterocycloalkyl optionally having an unsubstituted methylene group replaced by CO;
R³ is H, cycloalkyl, heterocycloalkyl, aryl, or heteroaryl, each cycloalkyl, heterocycloalkyl, aryl, and heteroaryl optionally substituted with one or more substituents selected from halo, alkyl, amino, nitro, cyano, alkoxyl, cycloalkyl, heterocycloalkyl, aryl, and heteroaryl, each optional substituent cycloalkyl, heterocycloalkyl, aryl, and heteroaryl optionally further substituted with one or more substituents selected from alkyl, amino, nitro, cyano, and alkoxyl;
R⁴ is H, alkyl, or NR⁶R⁷;
R⁵ is H alkyl;
R⁶ is H, cycloalkyl, heterocycloalkyl, aryl, or heteroaryl, each cycloalkyl, heterocycloalkyl, aryl, and heteroaryl optionally substituted with one or more substituents selected from halo, alkyl, amino, nitro, cyano, alkoxyl, cycloalkyl, heterocycloalkyl, aryl, and heteroaryl, each optional substituent heterocycloalkyl, aryl, and heteroaryl optionally further substituted with one or more substituents selected from alkyl, amino, nitro, cyano, alkoxyl, cycloalkyl, heterocycloalkyl, aryl, and heteroaryl, each further optional substituent cycloalkyl, heterocycloalkyl, aryl, and heteroaryl optionally substituted with one or more substituents selected from alkyl, amino, nitro, cyano, and alkoxyl; and
R⁷ is H or alkyl;
or a pharmaceutically acceptable salt, solvate, or stereoisomer thereof.

4. The method of aspect 2 or 3, wherein R¹ is C₁-C₄ alkyl optionally substituted with phenyl, the phenyl optionally further substituted with fluorine.

5. The method of any one of aspects 2-4, wherein R¹ is isopropyl, 4-fluorobenzyl, or 2-butyl.

6. The method of any one of aspects 2-5, wherein R² is —L—NR⁵—(CH₂)ₘ—X³.

7. The method of aspect 6, wherein L is CO.

8. The method of any one of aspects 2-7, wherein R² is —CO—NH—(CH₂)-heterocycloalkyl, the heterocycloalkyl optionally substituted with alkyl and optionally having an unsubstituted methylene group replaced by CO.

9. The method of any one of aspects 2-8, wherein R² is

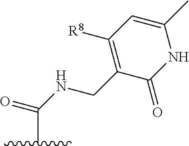

wherein R⁸ is methyl or n-propyl.

10. The method of any one of aspects 2-9, wherein R³ is heteroaryl optionally substituted with heterocycloalkyl, the heterocycloalkyl optionally further substituted with alkyl.

11. The method of any one of aspects 2-10, wherein R³ is pyridinyl substituted with piperazinyl, the piperazinyl optionally further substituted with alkyl.

12. The method of any one of aspects 2-11, wherein R³ is

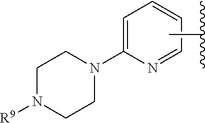

wherein R⁹ is H, methyl, or isopropyl.

13. The method of any one of aspects 2-12, wherein R⁴ is methyl or NH-(heterocycloalkyl), the heterocycloalkyl optionally substituted with alkyl, the alkyl optionally further substituted with aryl, the aryl optionally further substituted with alkoxyl.

14. The method of any one of aspects 2-13, wherein R⁴ is NH-(piperidinyl)-(alkyl)-(phenyl)-alkoxyl.

15. The method of any one of aspects 2-14, wherein R⁴ is

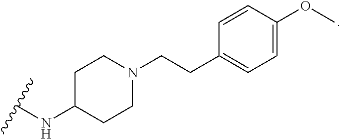 [SMILES: *NC1CCN(CCc2ccc(OC)cc2)CC1]

16. The method of aspects 2 or 3, wherein

R¹ is isopropyl, 4-fluorobenzyl, or 2-butyl;

### R² is

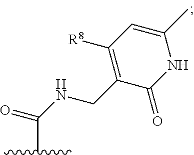

### R³ is

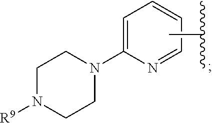

### R⁴ is

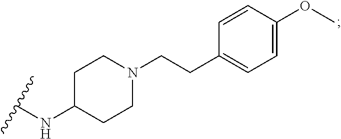 [SMILES: *NC1CCN(CCc2ccc(OC)cc2)CC1]

R⁸ is methyl or n-propyl; and
R⁹ is H, methyl, or isopropyl.

17. The method of aspect 2 or 3, wherein the compound is a compound of Formula (II):

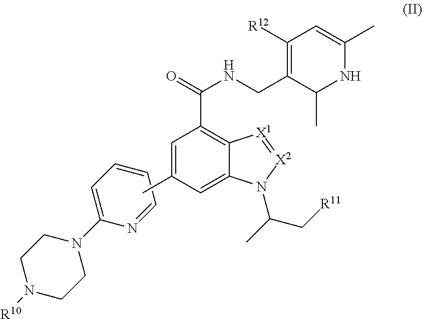

wherein X¹ and X² are each CR⁴ or X¹ is CR⁴ and X² is N; R⁴ is H or methyl; R¹⁰ is H, methyl, ethyl, or propyl; R¹¹ is H or methyl; and R¹² is methyl, ethyl, or propyl.

18. The method of aspect 2 or 3, wherein the compound is

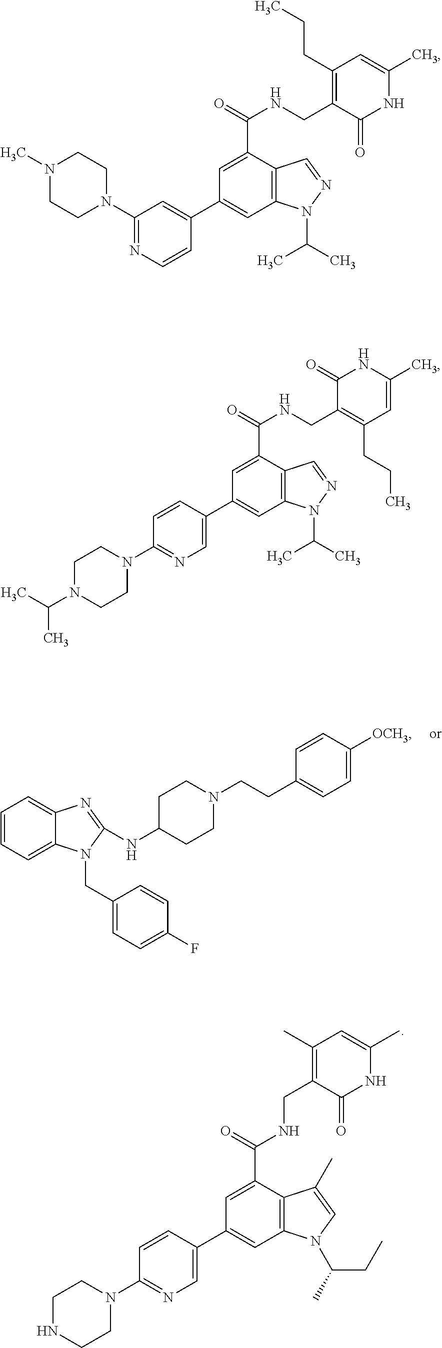 [SMILES: CCCc1cc(C)[nH]c(=O)c1CNC(=O)c1cc(-c2ccnc(N3CCN(C)CC3)c2)cc2c1cnn2C(C)C ; CCCc1cc(C)[nH]c(=O)c1CNC(=O)c1cc(-c2ccc(N3CCN(C(C)C)CC3)nc2)cc2c1cnn2C(C)C ; COc1ccc(CCN2CCC(Nc3nc4ccccc4n3Cc3ccc(F)cc3)CC2)cc1 ; CC[C@H](C)n1cc(C)c2c(C(=O)NCc3c(C)cc(C)[nH]c3=O)cc(-c3ccc(N4CCNCC4)nc3)cc21]

19. The method of any one of aspects 1-18, wherein the viral infection involves reactivation of a virus after latency in the host.

20. The method of any one of aspects 1-19, wherein the viral infection is due to a herpesvirus or adenovirus.

21. The method of any one of aspects 1-20, wherein the viral infection is acute.

22. The method of any one of aspects 1-21, wherein the compound is administered topically.

23. A method of improving the therapeutic effect of a pharmaceutical composition, the method comprising adding to the pharmaceutical composition a compound of Formula (I):

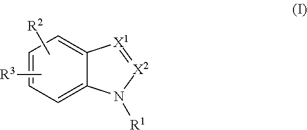

wherein
X¹ and X² are each CR⁴, X¹ is N and X² CR⁴, or X¹ is CR⁴ and X² is N;
R¹ is alkyl optionally substituted with one or more substituents selected from cycloalkyl, heterocycloalkyl, aryl, and heteroaryl, each substituent optionally further substituted with one or more substituents selected from halo, alkyl, amino, intro, cyano, and alkoxyl;
R² is H or —L—NR⁵—(CH₂)ₘ—X³,

### L is SO₂ or CO,

m is 0 to 3,
X³ is H, cycloalkyl, heterocycloalkyl, aryl, or heteroaryl, each cycloalkyl, heterocycloalkyl, aryl, and heteroaryl optionally substituted with one or more substituents selected from halo, alkyl, amino, nitro, cyano, and alkoxyl, the cycloalkyl and heterocycloalkyl optionally having an unsubstituted methylene group replaced by CO;
R³ is H, cycloalkyl, heterocycloalkyl, aryl, or heteroaryl, each cycloalkyl, heterocycloalkyl, aryl, and heteroaryl optionally substituted with one or more substituents selected from halo, alkyl, amino, nitro, cyano, alkoxyl, cycloalkyl, heterocycloalkyl, aryl, and heteroaryl, each optional substituent cycloalkyl, heterocycloalkyl, aryl, and heteroaryl optionally further substituted with one or more substituents selected from alkyl, amino, nitro, cyano, and alkoxyl;
R⁴ is H, alkyl, or NR⁶R⁷;
R⁵ is H or alkyl;
R⁶ is H, cycloalkyl, heterocycloalkyl, aryl, or heteroaryl, each cycloalkyl, heterocycloalkyl, aryl, and heteroaryl optionally substituted with one or more substituents selected from halo, alkyl, amino, nitro, cyano, alkoxyl, cycloalkyl, heterocycloalkyl, aryl, and heteroaryl, each optional substituent alkyl, cycloalkyl, heterocycloalkyl, aryl, and heteroaryl optionally further substituted with one or more substituents selected from alkyl amino, nitro, cyano, alkoxyl, cycloalkyl, heterocycloalkyl, aryl, and heteroaryl, each further optional substituent cycloalkyl, heterocycloalkyl, aryl, and heteroaryl optionally substituted with one or more substituents selected from alkyl, amino, nitro, cyano, and alkoxyl; and
R⁷ H or alkyl;
or a pharmaceutically acceptable salt, solvate, or stereoisomer thereof.

24. The method of aspect 23, wherein R¹ is C₁-C₄ alkyl optionally substituted with phenyl, the phenyl optionally further substituted with fluorine.

25. The method of aspect 23 or 24, wherein R¹ is isopropyl, 4-fluorobenzyl, or 2-butyl.

26. The method of any one of aspects 23-25, wherein R² is —L—NR⁵—(CH₂)ₘ—X³.

27. The method of aspect 26, wherein L is CO.

28. The method of any one of aspects 23-27, wherein R² is —CO—NH—(CH₂)-heterocycloalkyl, the heterocycloalkyl optionally substituted with alkyl and optionally having an unsubstituted methylene group replaced by CO.

29. The method of any one of aspects 23-28, wherein R² is

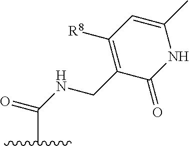

wherein R⁸ is methyl or n-propyl.

30. The method of any one of aspects 23-29, wherein R³ is heteroaryl optionally substituted with heterocycloalkyl, the heterocycloalkyl optionally further substituted with alkyl.

31. The method of any one of aspects 23-30, wherein R³ is pyridinyl substituted with piperazinyl, the piperazinyl optionally further substituted with alkyl.

32. The method of any one of aspects 23-31, wherein R³ is

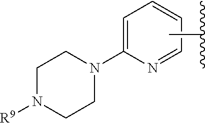

wherein R⁹ is H, methyl, or isopropyl.

33. The method of any one of aspects 23-32, wherein R⁴ is methyl or NH-(heterocycloalkyl), the heterocycloalkyl optionally substituted with alkyl, the alkyl optionally further substituted with aryl, the aryl optionally further substituted with alkoxyl.

34. The method of any one of aspects 23-33, wherein R⁴ is NH-(piperidinyl)-(alkyl)-(phenyl)-alkoxyl.

35. The method of any one of aspects 23-34, wherein R⁴ is

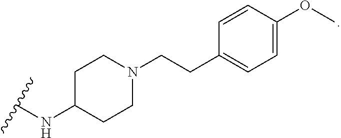 [SMILES: *NC1CCN(CCc2ccc(OC)cc2)CC1]

36. The method of aspects 3 or 24, wherein

R¹ is isopropyl, 4-fluorobenzyl, or 2-butyl;

### R² is

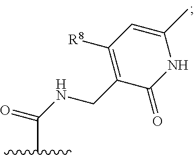

### R³ is

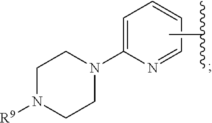

### R⁴ is

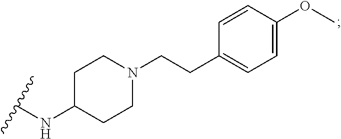 [SMILES: *NC1CCN(CCc2ccc(OC)cc2)CC1]

R⁸ is methyl or n-propyl; and
R⁹ is H, methyl, or isopropyl.

37. The method of aspect 23 or 24, wherein the compound is a compound of Formula (II):

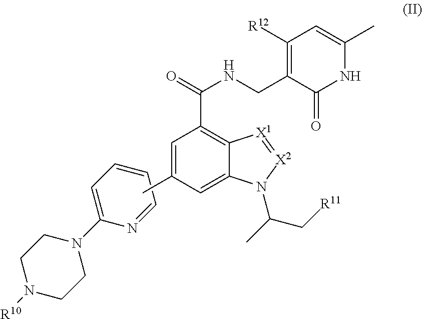

wherein X¹ and X² are each CR⁴ or X¹ is CR⁴ and X² is N; R⁴ is H or methyl; R¹⁰ is H, methyl, ethyl, or propyl; R¹¹ is H or methyl; and R¹² is methyl, ethyl, or propyl.

38. The method of aspect 23 or 24, wherein the compound is

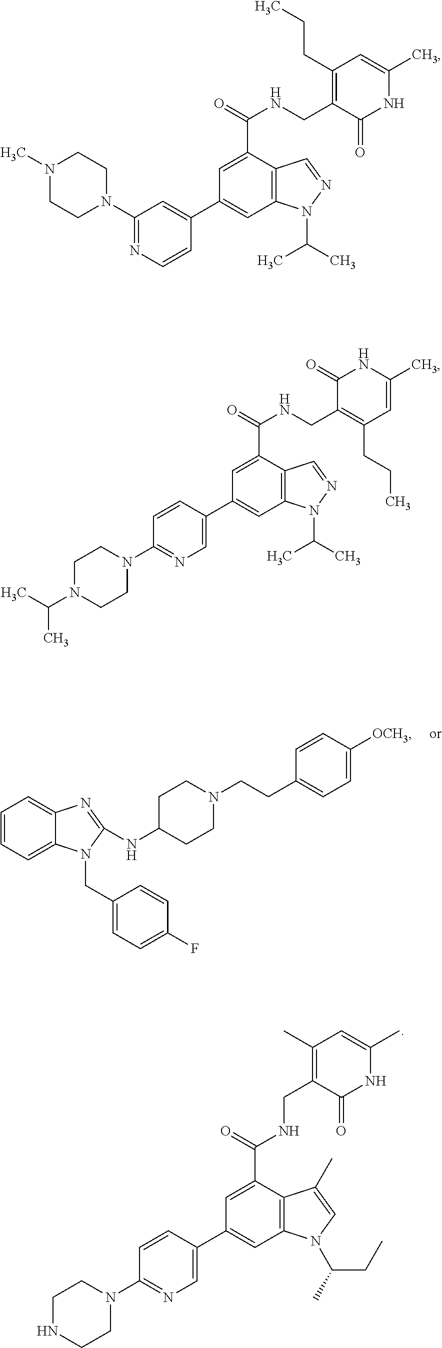 [SMILES: CCCc1cc(C)[nH]c(=O)c1CNC(=O)c1cc(-c2ccnc(N3CCN(C)CC3)c2)cc2c1cnn2C(C)C ; CCCc1cc(C)[nH]c(=O)c1CNC(=O)c1cc(-c2ccc(N3CCN(C(C)C)CC3)nc2)cc2c1cnn2C(C)C ; COc1ccc(CCN2CCC(Nc3nc4ccccc4n3Cc3ccc(F)cc3)CC2)cc1 ; CC[C@H](C)n1cc(C)c2c(C(=O)NCc3c(C)cc(C)[nH]c3=O)cc(-c3ccc(N4CCNCC4)nc3)cc21]

It shall be noted that the preceding are merely examples of embodiments. Other exemplary embodiments are apparent from the entirety of the description herein. It will also be understood by one of ordinary skill in the art that each of these embodiments may be used in various combinations with the other embodiments provided herein.

The following examples further illustrate the invention but, of course, should not be construed as in any way limiting its scope.

### EXAMPLE 1

This example demonstrates reduced lytic HSV IE expression with use of inhibitors that target distinct domains of EZH2 and EZH1, in accordance with embodiments of the invention.

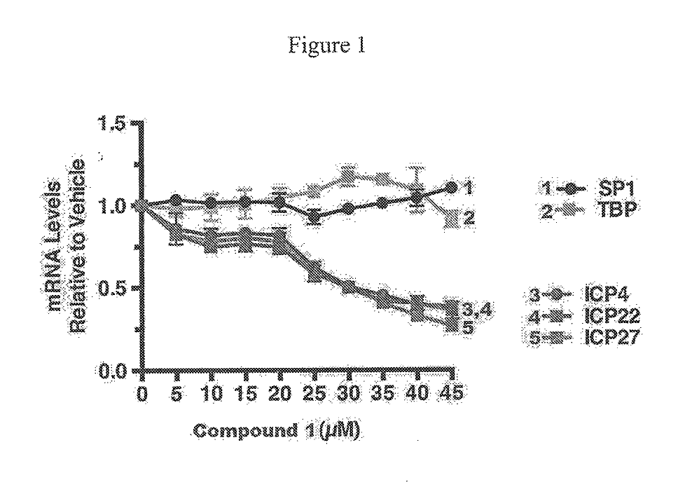

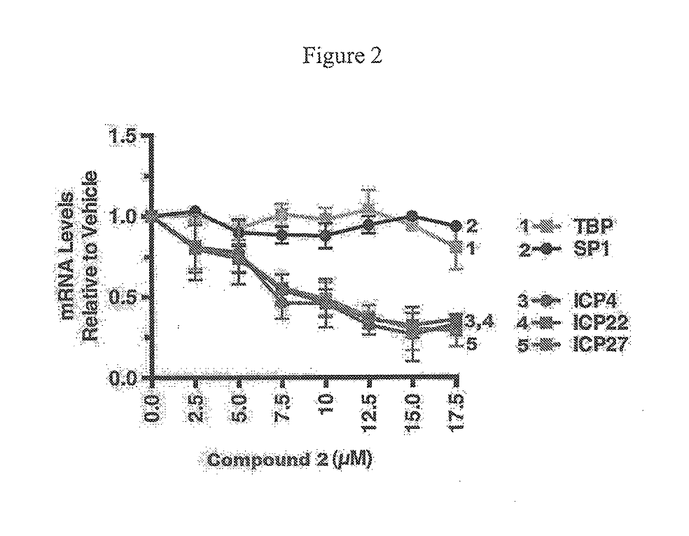

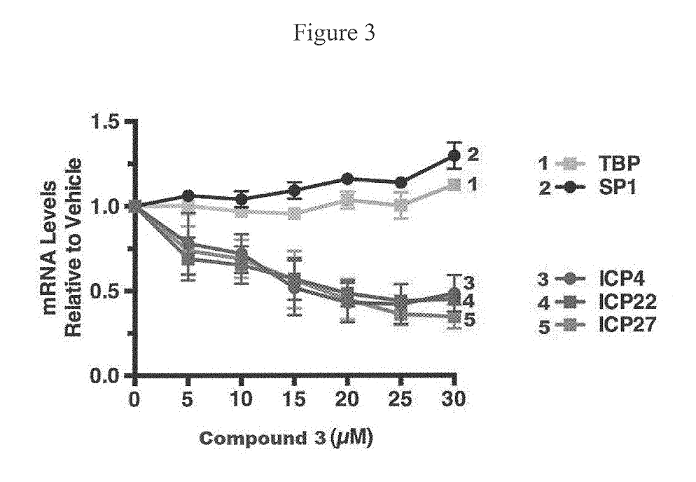

HFF cells were treated with increasing concentrations of EZH1/2 catalytic inhibitor (compound 1 or 2), an inhibitor that blocks the interaction between the polycomb group proteins EZH1/2 and EED (compound 3), or DMSO (vehicle) for 5-hrs. The HFF cells were then infected with HSV-1 [2.0 PFU (plaque-forming units) per cell] for 1.5-hrs in the presence of inhibitor or DMSO. The results are shown in FIGS. 1-3. In the figures, the levels of HSV viral IE (ICP4, ICP22, and ICP27) and cellular control (TBP and SP1) mRNAs are expressed relative to cells treated with DMSO (vehicle).

### EXAMPLE 2

This example demonstrates an EZH1/2 catalytic inhibitor suppresses HSV IE expression at high MOI, in accordance with embodiments of the invention.

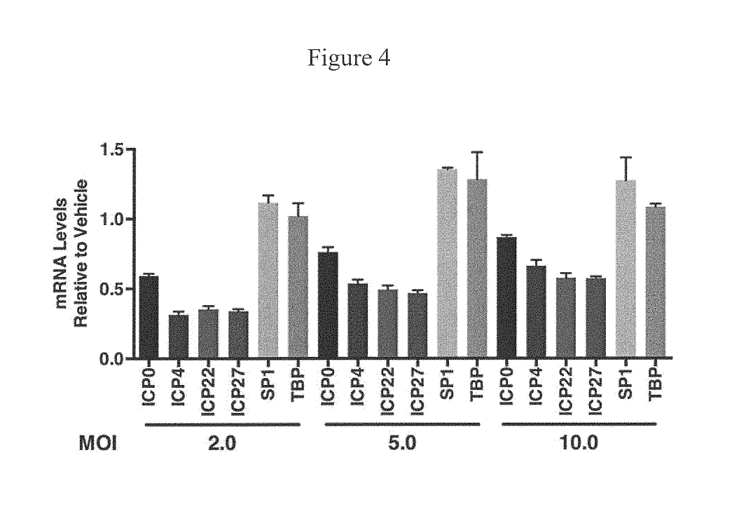

HFF cells were treated with the EZH1/2 catalytic inhibitor compound 4 (30 μM) or DMSO (vehicle) for 5-hrs. The HFF cells were then infected with HSV-1 (2.0, 5.0, and 10.0 PFU per cell) for 1.5-hrs in the presence of the inhibitor or DMSO. The results are shown in FIG. 4. The levels of HSV viral IE (ICP0, ICP4, ICP22, and ICP27) and cellular controls (SP1 and TBP) mRNAs are expressed relative to cells treated with DMSO (vehicle).

### EXAMPLE 3

This example demonstrates the EZH1/2 catalytic inhibitors have no impact on HSV-1 cellular and nuclear entry, in accordance with embodiments of the invention.

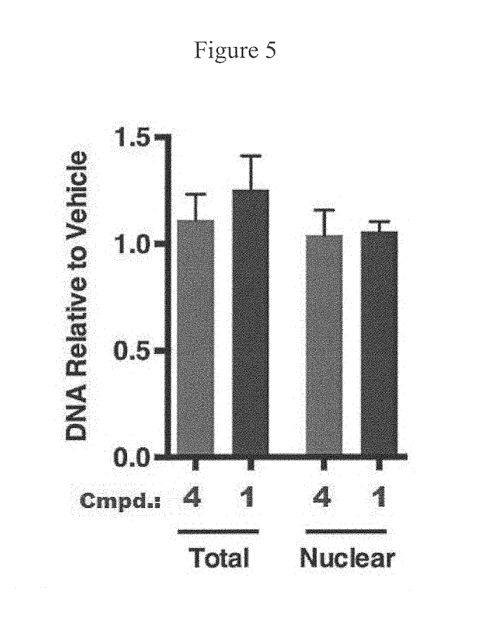

HFF cells were treated with the EZH1/2 catalytic inhibitor compound 1 (35 μM), compound 4 (30 μM), or DMSO (vehicle) for 5-hrs. The HFF cells were then infected with HSV-1 (2.0 PFU per cell) for 1.5-hrs in the presence of the inhibitors or DMSO (vehicle). The results are shown in FIG. 5. The levels of HSV viral DNA isolated from total and nuclear cellular fractions are expressed as ratios relative to DMSO (vehicle). Both compounds have no impact on HSV-1 viral entry (total and nuclear), suggesting the block in viral gene expression shown in previous Examples is through transcriptional repression.

### EXAMPLE 4

This example demonstrates repression of HSV by an EZH1/2 inhibitor occurs prior to the establishment of IE mRNA expression, in accordance with embodiments of the invention.

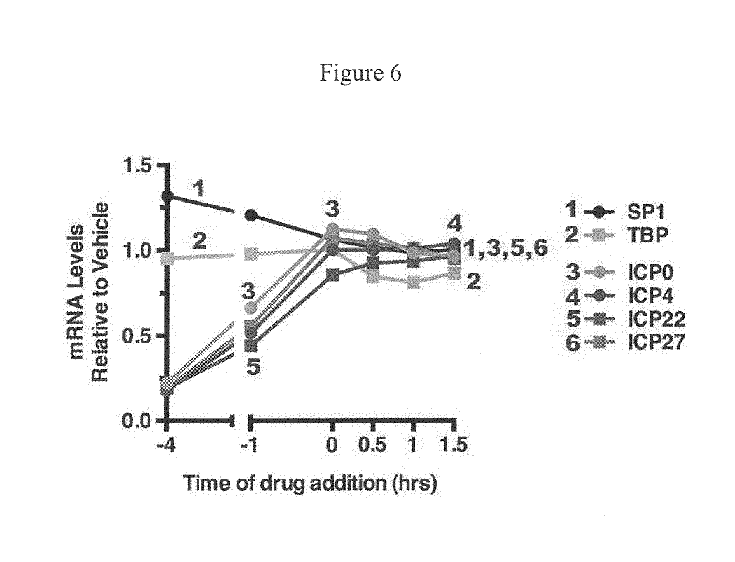

HFF cells were treated with the EZH1/2 catalytic inhibitor compound 4 (30 μM) or DMSO (vehicle) for the indicated duration in FIG. 6. HFF cells were then infected with HSV-1 (2.0 PFU per cell) for 1.5-hrs in the presence of compound 4 or DMSO (vehicle). The levels of HSV viral IE (ICP0, ICP4, ICP22, and ICP27) and cellular control (SP1 and TBP) mRNAs are expressed relative to cells treated with DMSO (vehicle).

### EXAMPLE 5

This example demonstrates the EZH1/2 inhibitors block the spread of HSV infection, in accordance with embodiments of the invention.

HFF cells were mock or infected with HSV-1 (MOI 0.01) for 8.5-hrs to allow one round of the viral replication program. The HFF cells were then treated with EZH1/2 inhibitor (compound 1 or compound 4 at 30 μM), viral DNA polymerase inhibitor (ACV at 100 μM), or JMJD3 inhibitor (ML324 at 50 μM) for an additional 12.5-hrs. Cells were paraformaldehyde fixed, permeabilized, and stained for the viral E gene UL29 and actin. The viral E protein UL29 was used as a marker for the spread of viral infection. Treatment with EZH1/2 compounds block the spread of HSV to adjacent cells.

### EXAMPLE 6

This example demonstrates the EZH1/2 catalytic inhibitors suppress hCMV mRNA expression, in accordance with embodiments of the invention.

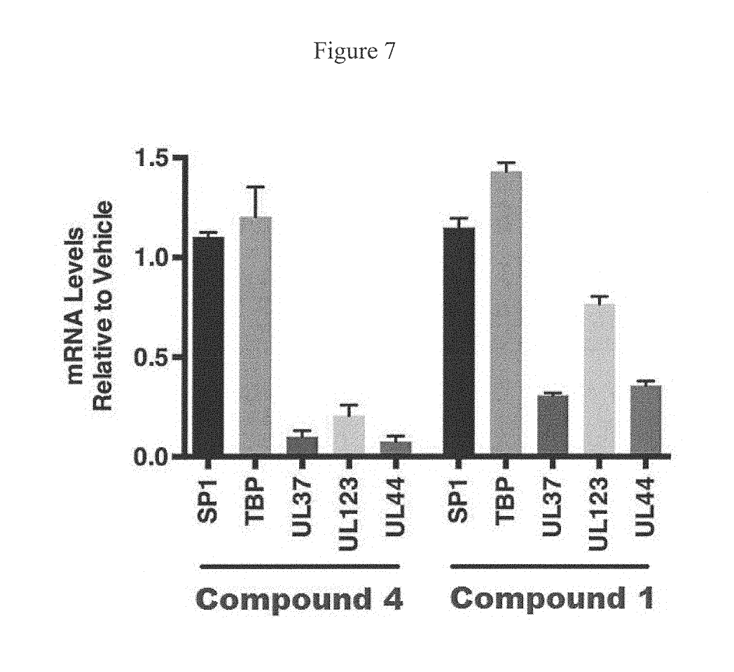

HFF cells were treated with EZH1/2 catalytic inhibitor compound 1 (35 μM), compound 4 (30 μM), or DMSO (vehicle) for 5-hrs. HFF cells were then infected with hCMV (0.5 PFU per cell) for 2-hrs in the presence of the inhibitor or DMSO (vehicle). FIG. 7 shows the results. The levels of hCMV viral IE (UL37, UL123), E (UL44) and cellular control (SP1 and TBP) mRNAs are expressed relative to cells treated with DMSO (vehicle).

### EXAMPLE 7

This example demonstrates the EZH1/2 catalytic inhibitors suppress ADV-5 mRNA expression, in accordance with embodiments of the invention.

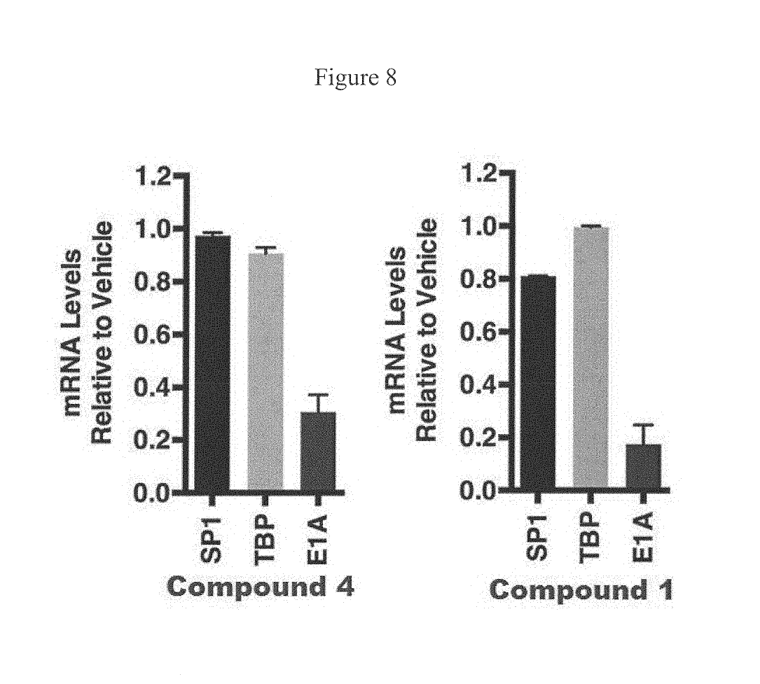

HFF cells were treated with EZH1/2 catalytic inhibitor compound 1 (35 μM), compound 4 (30 μM), or DMSO (vehicle) for 5-hrs. HFF cells were then infected with ADV-5 (200 PFU per cell) for 3-hrs in the presence of the inhibitor or DMSO (vehicle). The results are at FIG. 8. The levels of ADV-5 viral E gene E1A and cellular control (SP1 and TBP) mRNAs are expressed relative to cells treated with DMSO (vehicle).

### EXAMPLE 8

This example demonstrates the EZH1/2 catalytic inhibitors have no impact in cells that are IFNβ and IRF3 deficient, in accordance with embodiments of the invention.

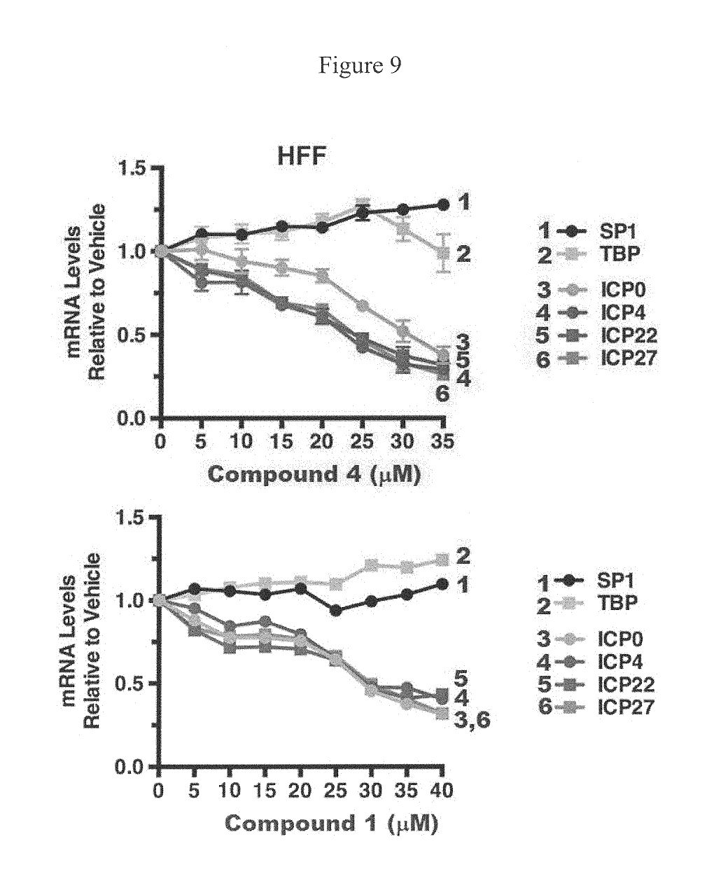

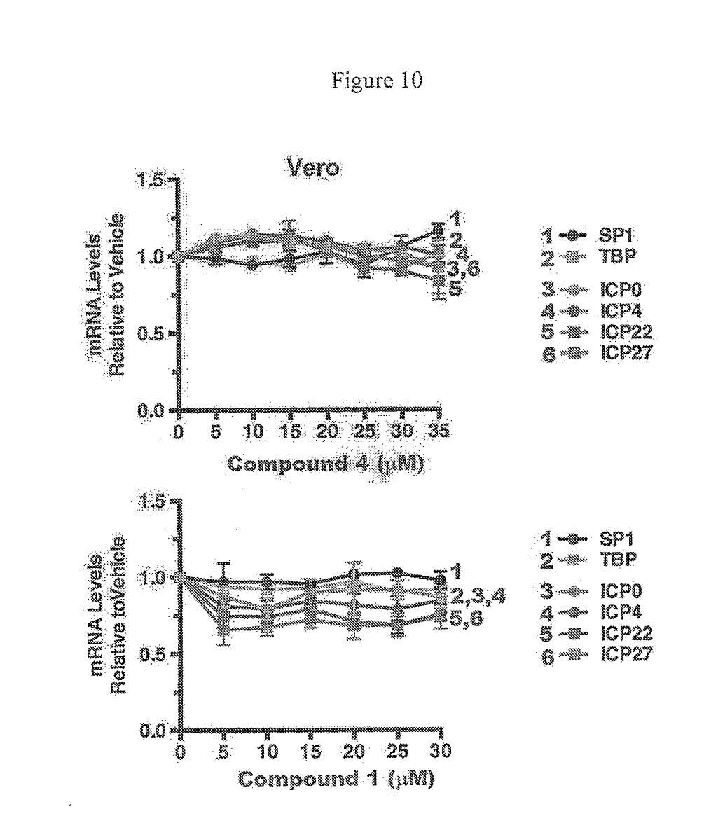

HFF (wild-type) and Vero (IFN-β null, IRF3 deficient) cells were treated with the indicated concentration of EZH1/2 catalytic inhibitor (compound 1 or 4) or DMSO (vehicle) for 5-hrs. HFF and Vero cells were then infected with HSV-1 (2.0 PFU per cell) for 1.5-hrs in the presence of inhibitor or DMSO. The results are shown in FIGS. 9 and 10. The levels of HSV viral IE (ICP0, ICP4, ICP22, and ICP27) and cellular control (SP1 and TBP) mRNAs are expressed relative to cells treated with DMSO (vehicle). The lack of antiviral activity in Vero as compared to HFF (wild-type) cells suggests that EZH1/2 regulates innate IFN signaling pathways.

### EXAMPLE 9

This example demonstrates EZH1/2 is a negative regulator of a subset of genes involved in innate interferon signaling, in accordance with embodiments of the invention.

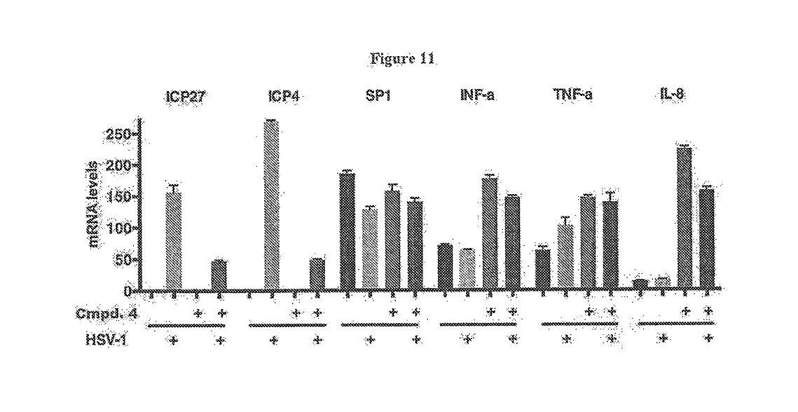

HFF cells were treated with EZH1/2 catalytic inhibitor compound 4 (30 μM) or DMSO for 5-hrs. HFF cells were then mock or infected with HSV-1 (2.0 PFU per cell) for 1.5-hrs in the presence of inhibitor or DMSO. The results are shown in FIG. 11. The levels of HSV viral IE (ICP4 and ICP27), control (SP1), and cellular innate interferon signaling (IFN-α, TNF-α, IL-8) mRNAs are expressed as absolute levels (absolute copies). EZH1/2 inhibitor compound 4 represses HSV viral IE expression with no impact on cellular control SP1. Compound 4 induces the expression of key innate antiviral signaling molecules IFN-α, TNF-α, and IL-8, suggesting that EZH1/2 is a negative regulator of a subset of antiviral genes.

### EXAMPLE 10

This example demonstrates increased duration of pretreatment with an EZH1/2 inhibitor enhances the HSV antiviral activity of these compounds, in accordance with embodiments of the invention.

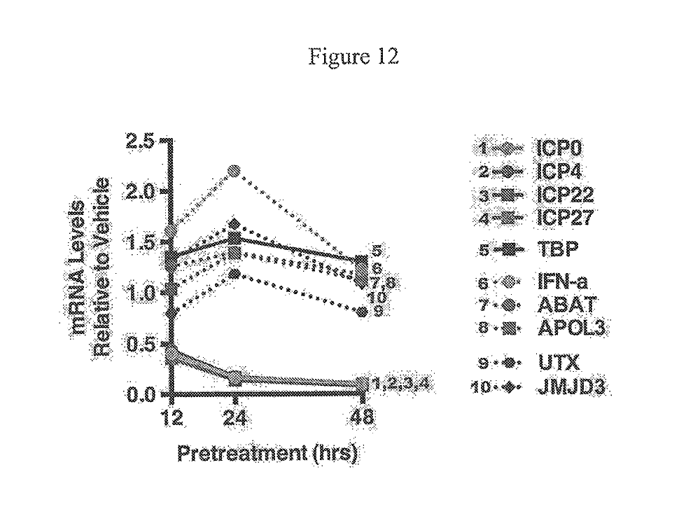

HFF cells were treated with the EZH1/2 catalytic inhibitor compound 4 (5 μM) or DMSO for the indicated time as shown in FIG. 12. The HFF cells were then infected with HSV-1 (2.0 PFU per cell) for 1.5-hrs in the presence of inhibitor or DMSO (vehicle). The levels of HSV viral IE (ICP0, ICP4, ICP22, and ICP27), control (TBP, ABAT, APOL3, UTX, and JMJD3), and cellular innate interferon signaling (IFN-α) mRNAs are expressed as ratios relative to cells treated with DMSO (vehicle). In this Example compound 4 was decreased to 5 μM and the duration of pretreatment was increased to 12, 24, and 48-hrs. Increasing the duration of pretreatment with these compounds enhanced the suppression of HSV IE expression.

### EXAMPLE 11

This example demonstrates the inhibitors targeting distinct domains of EZH2 and EZH1 block HSV reactivation in the mouse ganglia explant model, in accordance with embodiments of the invention.

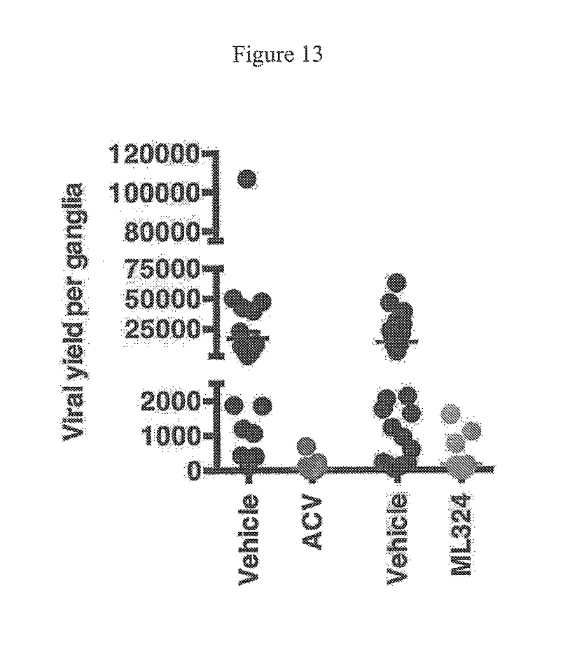

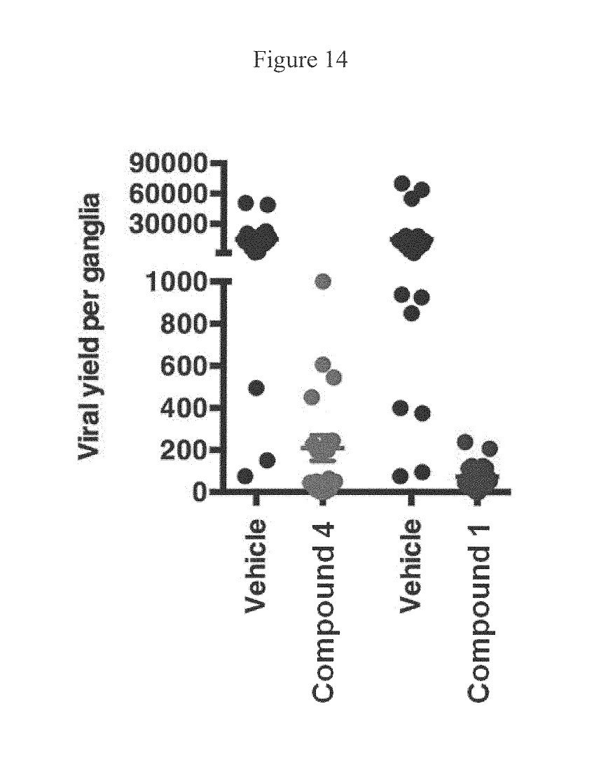

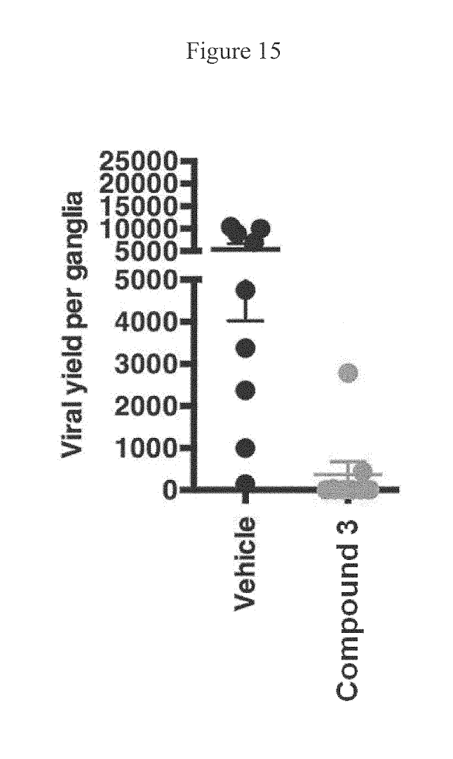

Trigeminal ganglia from HSV-1 latently infected mice were bisected. Half were explanted in media with control DMSO (vehicle), and the other half were explanted in media with ACV (100 μM), ML324 (50 μM), compound 1 (35 μM), compound 3 (30 μM), or compound 4 (30 μM) for 48-hrs to induce viral reactivation. Viral yields were determined by titrating on Vero cells. The results are presented in FIGS. 13-15. Each point represents the titer of one explanted trigeminal ganglia. Both EZH1/2 catalytic (compounds 1 and 4) and EZH1/2-EED interaction (compound 3) inhibitors block HSV reactivation from latency.

### EXAMPLE 12

This example demonstrates the EZH1/2 catalytic inhibitors suppress HSV DNA yields during viral reactivation, in accordance with embodiments of the invention.

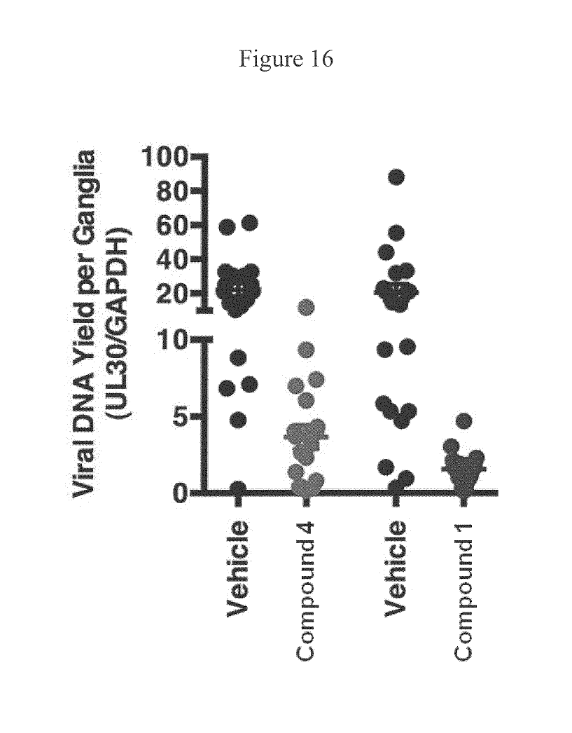

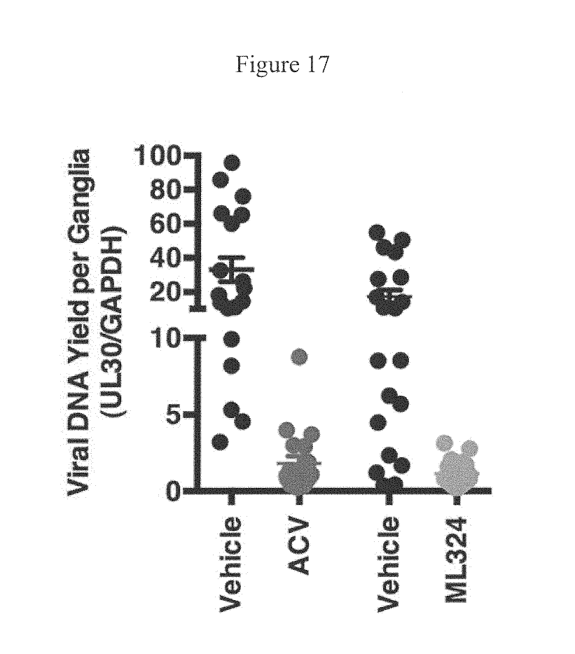

Trigeminal ganglia from HSV-1 latently infected mice were bisected and half explanted in media with control DMSO (vehicle) and the other half explanted in media with compound 1 (35 μM), compound 4 (30 μM), ACV (100 μM), or ML324 (50 μM) for 48-hrs to induce viral reactivation. The results are presented in FIGS. 16 and 17. Viral DNA yields (per ganglia) were determined by qPCR amplification of the viral ORF UL30 and normalized to the levels of cellular control GAPDH.

### EXAMPLE 13

This example demonstrates the suppression of EZH1/2 catalytic activity blocks HSV reactivation in individual and neuron clusters, in accordance with embodiments of the invention.

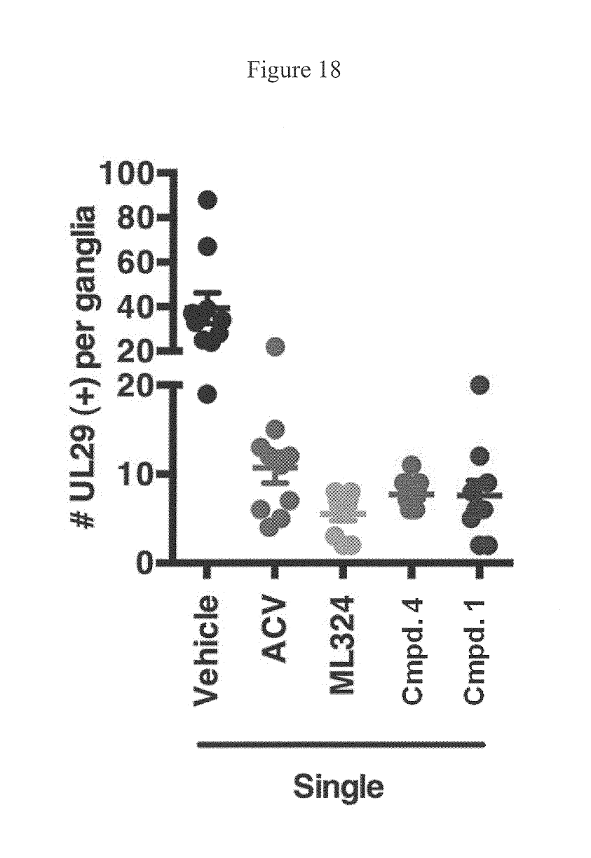

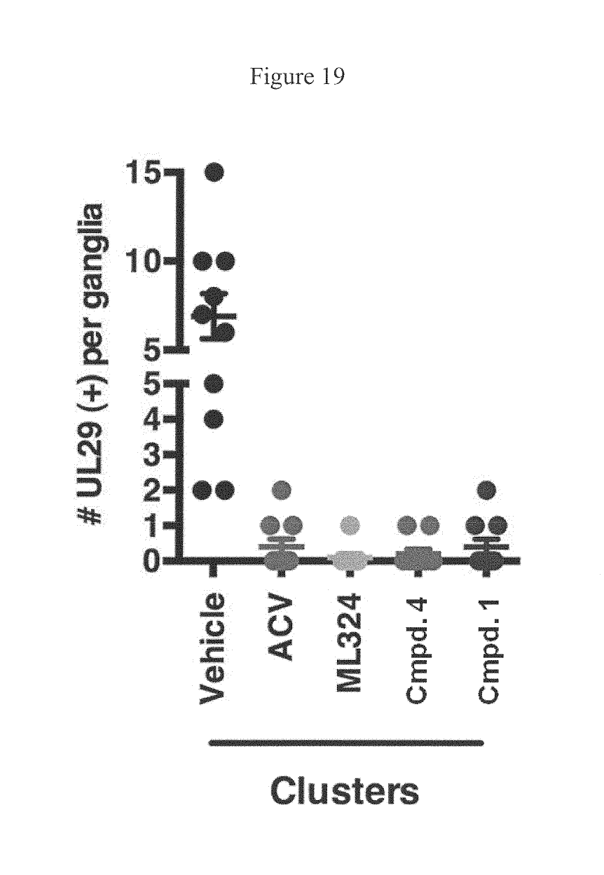

Trigeminal ganglia from HSV-1 latently infected mice were explanted in media with control DMSO (vehicle), compound 1 (35 μM), compound 4 (30 μM), ACV (100 μM), or ML324 (50 μM) for 48-hrs to induce viral reactivation. Trigeminal ganglia were fixed in paraformaldehyde and tissue sections were contained with anti-UL29 and DAPI. The HSV E gene UL29 (DNA single strand binding replication protein) was used a marker for viral reactivation. Tissue sections were scored for UL29+ cell clusters (clusters indicate viral spread from the primary neuron to surrounding cells) and for individual neurons representing the primary reactivation event (single) (FIGS. 18 and 19).

Compound 1, compound 4, ACV, and ML324 inhibitors reduced both primary reactivation and secondary spread of HSV in explanted trigeminal ganglia from latently infected mice.

### EXAMPLE 14

This example demonstrates an EZH1/2 inhibitor induces innate antiviral pathways, in accordance with embodiments of the invention.

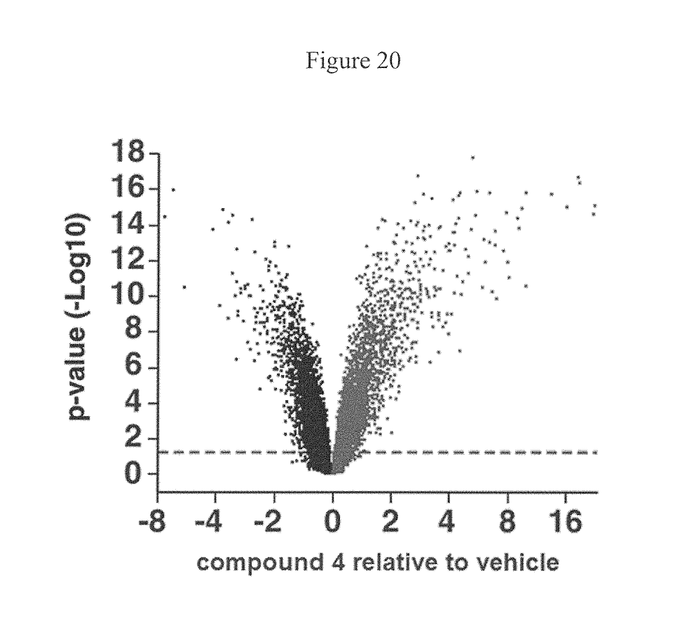

HFF cells were treated with compound 4 (30 μM) or DMSO (vehicle) for 4-hrs, and total cellular DNA was isolated. Microarray analysis identified 252 genes that were induced greater than 2 fold with compound 4 relative to vehicle (FIG. 20). Of those genes that were induced>2 fold, 212 genes (84%) are co-regulated by IFN and compound 4 treatment alone (Interferome, v2.01 from the Australian National Data Service (ANDS), see Rusinova et al., Nucleic Acids Research, 41 (database issue): D1040-D1046 (2013), incorporated herein by reference). In normal fibroblast cells (as opposed to established cell lines or transformed cells lines), 41 genes are co-regulated by IFN (16%) and compound 4 treatment alone. Ingenuity Pathway Analysis (QIAGEN, Venlo, Netherlands) identified multiple pro-inflammatory pathways induced with compound 4 treatment: IL6, IL17, TLR, HMGB1, and JAK/STAT signaling pathways.

These analyses indicate significant overlap in the induced expression of genes in cells treated with compound 4 with that of cells treated with INF.

### EXAMPLE 15

This example demonstrates an EZH1/2 inhibitor induces innate gene expression a mouse ganglia explants model, in accordance with embodiments of the invention.

Trigeminal ganglia from HSV-1 latently infected mice were explanted in media with control DMSO (vehicle) or in media with compound 4 (30 μM) for 12 hrs. Total cellular RNA was isolated and RNA-seq analysis identified the induction of cytokines, chemokines, and adhesion proteins involved in innate signaling and recruitment of immune effector cells. The results are in Table 1 below, where changes in gene expression are expressed as ratios of compound 4 relative to vehicle.

TABLE 1

| | | Fold Change (Cmpd. 4 |
|---|---|---|
| | Gene | relative to vehicle) |
| Cytokines | G-CSF | 6.16 |
| | GM-CSF | 5.51 |
| | IL6 | 3.61 |
| | LIF | 2.66 |
| | IL11 | 2.16 |
| | VEGF-c | 2.10 |
| Chemokines | CXCL1 | 6.30 |
| | CXCL2 | 5.11 |
| | CXCL3 | 2.68 |
| | CXCL5 | 3.78 |
| | CCL2 | 2.23 |
| Adhesion | SELE | 3.86 |
| | SELP | 3.16 |
| | ICAM1 | 2.51 |

Of those genes that were induced>2 fold with compound 4, 33 genes (69%) are co-regulated by IFN and compound 4 treatment alone (Interferome, v2.01 from the Australian National Data Service (ANDS)).

### EXAMPLE 16

This example demonstrates removal of EZH1/2 inhibitors prior to infection leads to the recovery of HSV IE expression, in accordance with embodiments of the invention.

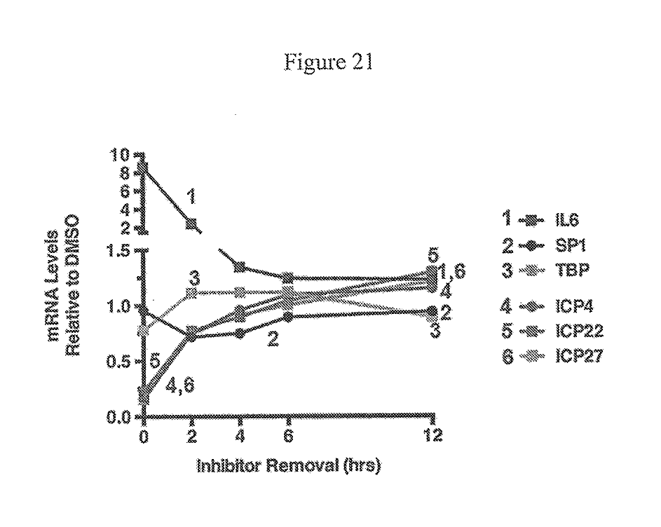

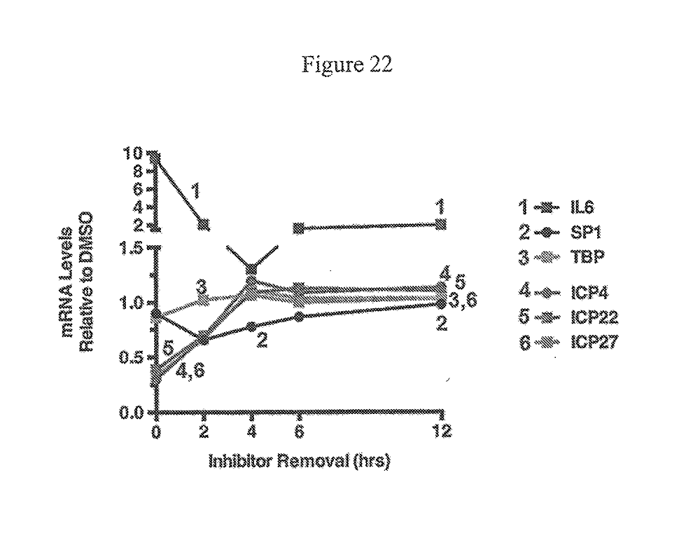

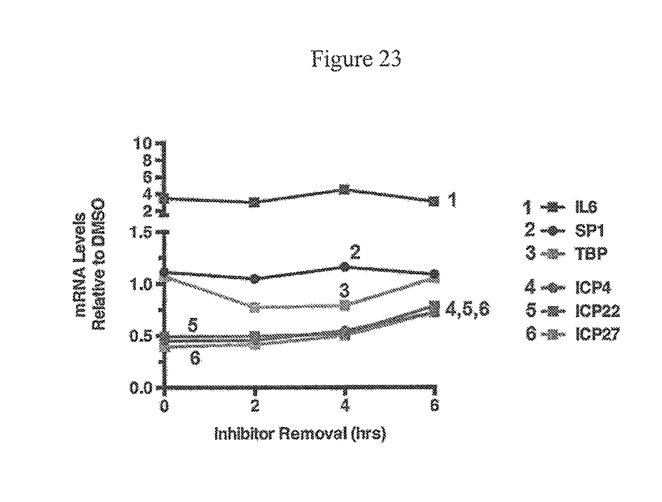

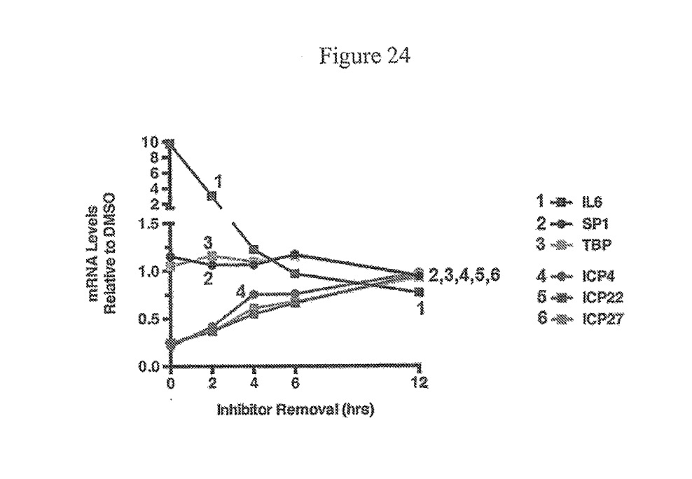

HFF cells were treated with inhibitors targeting the catalytic SET domain (compound 1, 35 μM: EZH2; compound 2, 15 μM: EZH2 and EZH1; compound 4, 30 μM: EZH2), EED-EZH2 and EED-EZH1 (compound 3, 30 μM), or DMSO (control) for 5-hrs. Cells were then washed with phosphate buffered saline (PBS) and replaced with media with no inhibitors for the indicated time in FIGS. 21-24 prior to infection with HSV-1 (2.0 PFU per cell) for 1.5-hrs in the absence of inhibitors.

The data suggest the impact of EZH1/2 inhibitors is readily reversed upon drug removal.

### EXAMPLE 17

This example demonstrates suppression of EZH1/2 catalytic activity reduces HSV reactivation, in sensory neurons, and spread, within sensory ganglia, in accordance with embodiments of the invention.

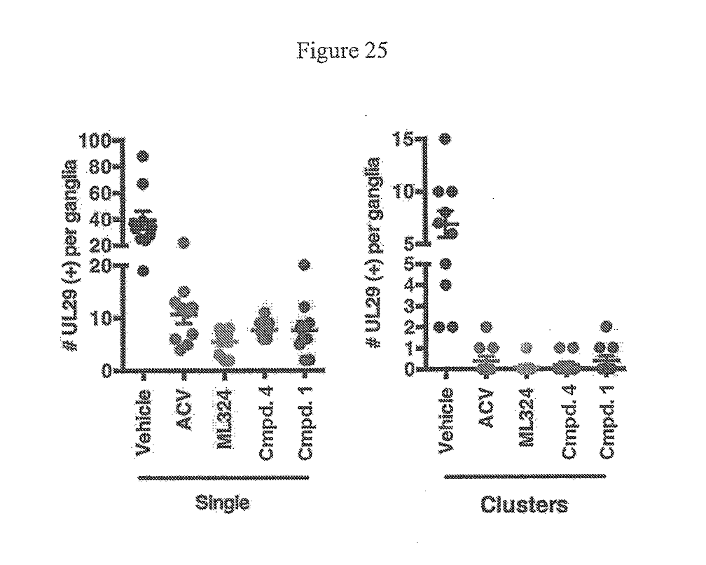

Trigeminal ganglia from HSV-1 latently infected mice were explanted in media with control DMSO (vehicle), ACV (100 μM), compound 1 (35 μM), compound 4 (30 μM), or ML324 (50 μM) for 48-hrs to induce viral reactivation. Trigeminal ganglia were fixed in paraformaldehyde and tissue sections were costained with anti-UL29 and DAPI. The HSV E gene UL29 (DNA single strand binding replication protein) was used a marker for viral reactivation. Tissue sections were scored for UL29+ cell clusters (clusters indicate viral spread from the primary neuron to surrounding cells) and for individual neurons representing the primary reactivation event (single) (FIG. 25).

EZH1/2 (compound 1, compound 4) and control (ACV, ML324) inhibitors reduced the number of single neurons and cluster-spread during explant-induced reactivation. EZH1/2 inhibitors reduce the number of primary neurons that undergo viral reactivation and reduce the spread of HSV within the sensory ganglia in a ganglia explant reactivation model system.

### EXAMPLE 18

This example demonstrates an EZH1/2 inhibitor induces the expression of innate gene expression in explanted ganglia, in accordance with embodiments of the invention.

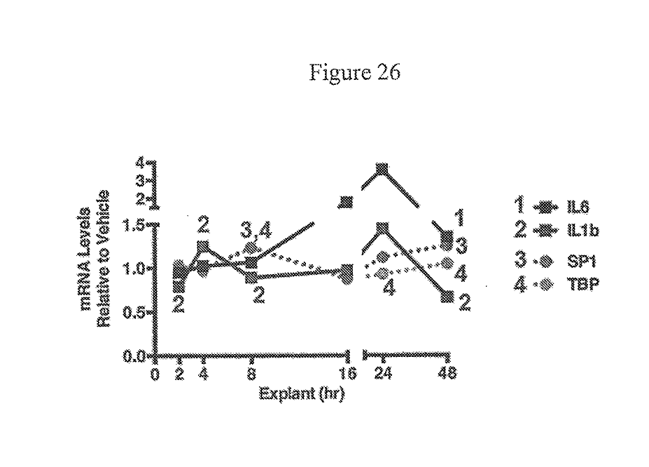

Trigeminal ganglia from Balb/c mice were explanted in media with control DMSO (vehicle) or compound 4 (30 μM) for the indicated duration in FIG. 26.

Similar to tissue culture cells, the EZH1/2 inhibitor induces the expression of innate immunity genes in cells of the sensory ganglia, indicating that the impacts of these inhibitors seen in tissue culture cells is also seen in tissues. This induction likely accounts for the decrease in HSV reactivation and spread in these tissues.

### EXAMPLE 19

This example demonstrates EZH1/2 inhibitors suppress primary infection in vivo, in accordance with embodiments of the invention.

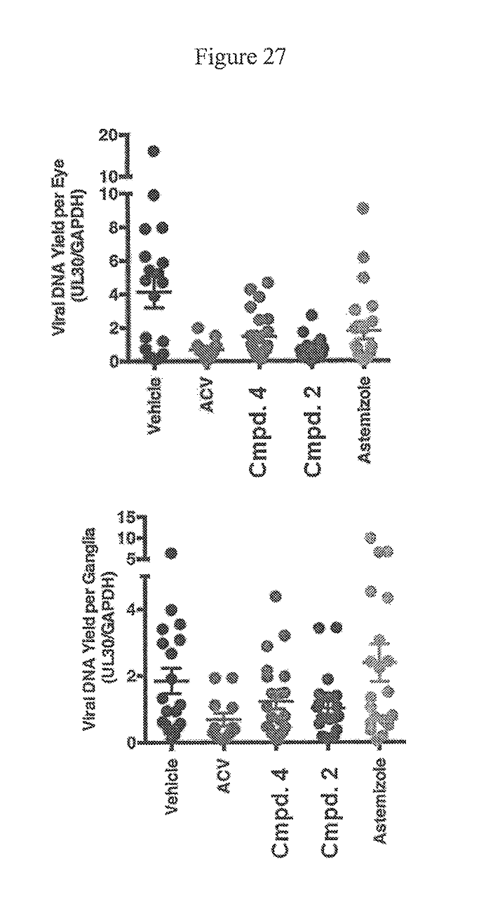

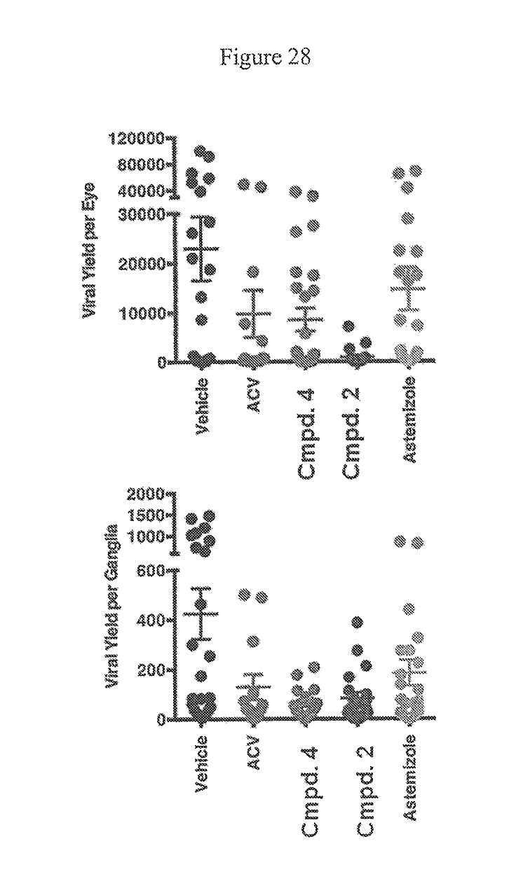

The eyes of Balb/c mice were infected with 2×10⁵ pfu of HSV-1 (strain F) per eye. Beginning on day 0.5, the eyes of mice were treated by application of 5 μl of either EZH2/1 inhibitors (compound 2: 1.5 μM, compound 3: 30 μM, compound 4: 30 μM), acyclovir (ACV: 30 μM) or vehicle control twice daily (twice per 24 period). On day 7, mice were sacrificed and the eyes and ganglia were isolated and viral DNA levels were determined through quantitative real-time PCR (FIG. 27) and viral yield (pfu) was determined by titering on Vero cells (FIG. 28).

Topical application of EZH1/2 inhibitors to the eyes of HSV infected mice (ocular infection) reduces the severity of the primary infection.

### EXAMPLE 20

This example demonstrates treatment with an EZH1/2 inhibitor enhances neutrophil recruitment to the site of viral infection in vivo, in accordance with embodiments of the invention.

The eyes of Balb/c mice were scarified and mock or infected with 2×10⁵ pfu of HSV-1 (strain F) per eye. Beginning on day 0.5, the eyes of mice were treated either with EZH1/2 inhibitor (compound 4: 30 μM), acyclovir (ACV: 30 μM), or vehicle control twice daily. On day 5, the eyes were fixed in paraformaldehyde and tissue sections were co-stained with anti-HSV-1, anti-Ly6G (neutrophil), and DAPI.

Recruitment/infiltration of neutrophils to the site of HSV infection upon treatment with EZH1/2 inhibitor compound 4 demonstrates the immune stimulation of these inhibitors in vivo.

### EXAMPLE 21

This example demonstrates inhibitors targeting distinct domains of EZH1/2 suppress lytic HSV protein expression, in accordance with embodiments of the invention.

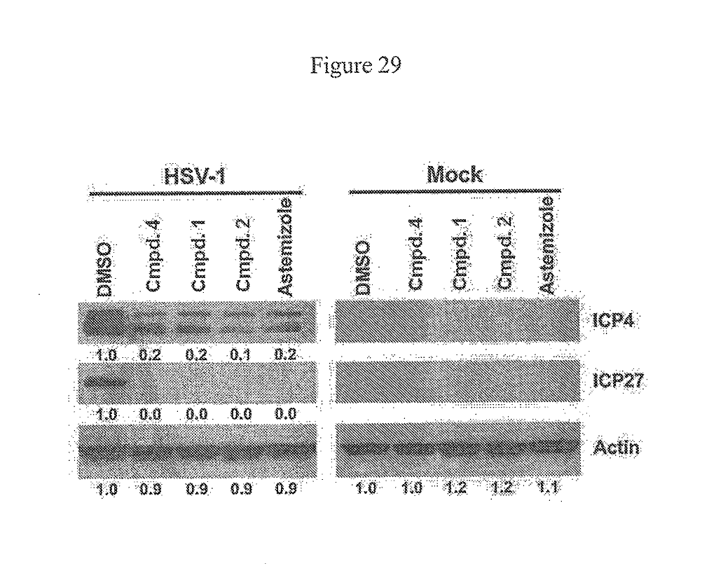

HFF cells were treated with inhibitors targeting the catalytic SET domain (compound 1: 40 μM, compound 2: 15 μM, compound 4: 30 μM), EED-EZH2 and EED-EZH1 (compound: 30 μM), or DMSO control for 5-hrs followed by infection with HSV-1 (2.0 PFU per cell) or mock for 2-hrs in the presence of inhibitors. Western blot of IE proteins (ICP4, ICP27) and the ratios to levels in DMSO treated cells are shown in FIG. 29 and are normalized to the actin-loading control.

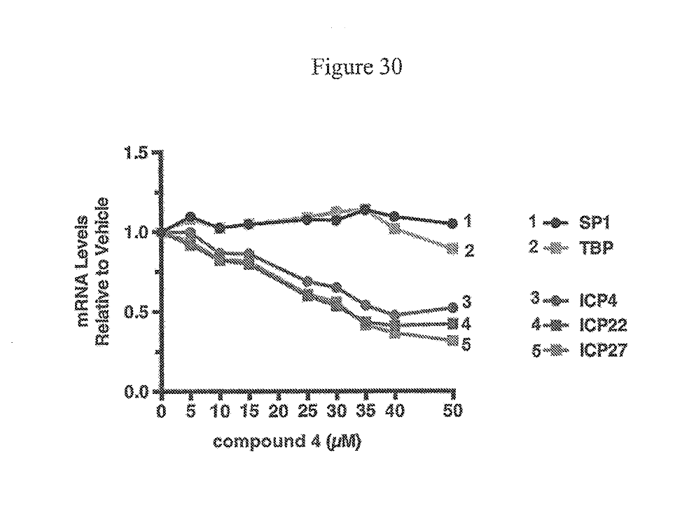

MRC-5 cells were treated with the indicated concentrations of EZH2 inhibitor compound 4 for 5-hrs followed by infection with HSV-1 (2.0 PFU per cell) for 1.5-hrs in the presence of inhibitor. Levels of HSV viral IE (ICP4, ICP22, ICP27) and cellular controls (SP1, TBP) mRNAs are shown in FIG. 30 and are expressed relative to cells treated with DMSO (vehicle).

HFF cells were mock or infected with HSV-1 (MOI 0.01) for 8.5-hrs to allow one round of the viral replication program. HFF cells were then treated with EZH1/2 (compound 1, compound 4), viral DNA polymerase (ACV), or JMJD3 (ML324) inhibitors for additional 12.5-hrs. Cells were paraformaldehyde fixed, permeabilized, and stained for the viral E gene UL29 and actin (Phalloidin). The viral E protein UL29 was used as a marker for the spread of viral infection. The data suggest that EZH1/2 inhibitors block the spread of HSV infection.

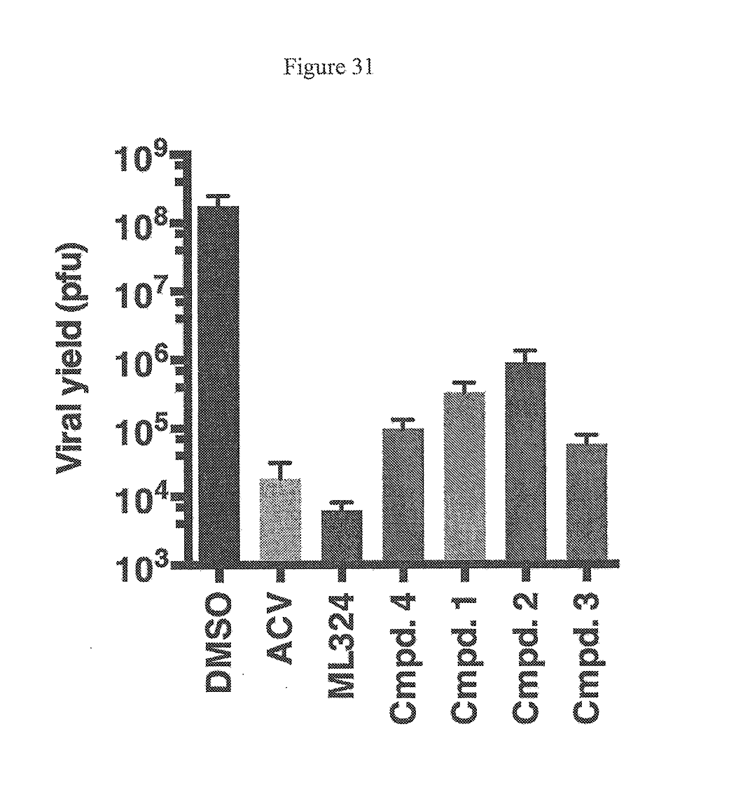

HFF cells were infected with HSV-1 (MOI 0.01) for 8-hrs to allow one round of the viral replication program. HFF cells were then treated with EZH1/2 (compound 1: 30 μM, compound 2: 15 μM, compound 3: 20 μM, compound 4: 25 μM), viral DNA polymerase (ACV: 100 μM), JMJD3 (ML324: 50 μM) inhibitors or DMSO (vehicle control) for additional 12-hrs (FIG. 31). Viral yields were determined titrating on Vero cells (plaque forming units: pfu).

Treatment of cells with EZH1/2 inhibitors that block the enzyme activity (catalytic inhibitor) or disrupt the EZH-PRC complex reduce the expression of the first wave of HSV genes (IE genes); suppress infection and spread of the infection to adjacent cells; and suppress viral yields.

### EXAMPLE 22

This example demonstrates inhibition of the Zika virus by compound 4, in accordance with embodiments of the invention.

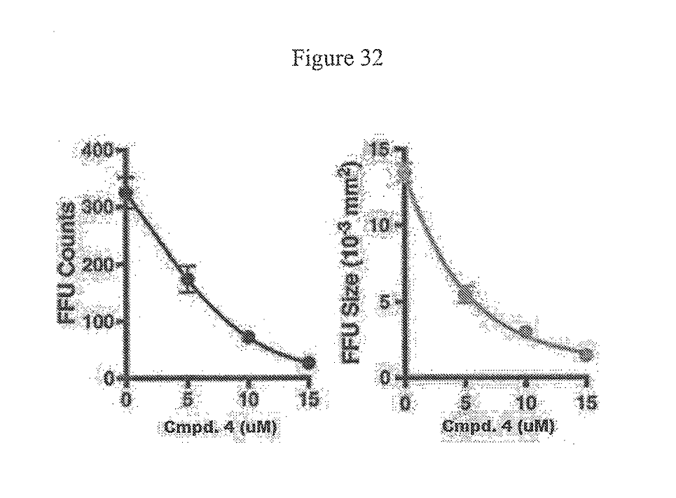

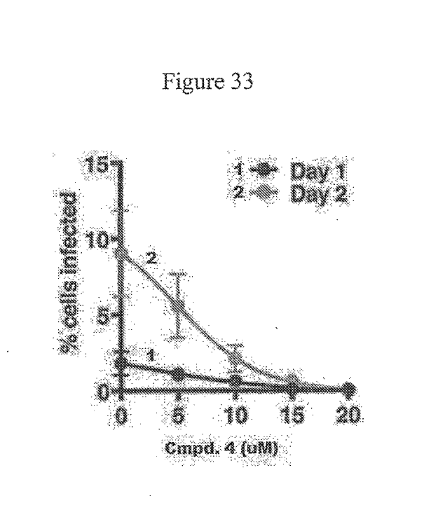

HFF cells were treated with compound 4 and infected with Zika virus (ZIKV), a member of the flavivirus family. Compound 4 significantly reduced both the number and size of ZIKV focus forming units (plaques) in a dose-dependent manner (FIG. 32). These results were further supported by compound 4-meditated reduction in the number of ZIKV infected cells at 1 and 2 dpi (days post infection) as measured by intracellular staining for ZIKV antigens (FIG. 33).

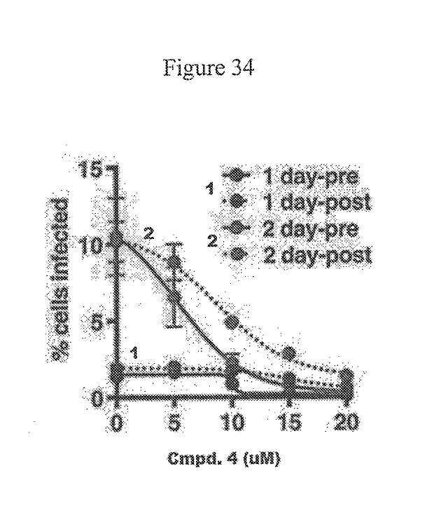

To determine if pretreatment was required to suppress ZIKV infection, cells were pretreated with compound 4 or were treated 3 h post ZIKV adsorption. While pretreatment was modestly more efficient at suppression of infection at lower compound 4 concentrations, it was not essential to effect significant suppression (FIG. 34).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two of more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Also, everywhere “comprising” (or its equivalent) is recited, the “comprising” is considered to incorporate “consisting essentially of” and “consisting of.” Thus, an embodiment “comprising” (an) element(s) supports embodiments “consisting essentially of” and “consisting of” the recited element(s). Everywhere “consisting essentially of” is recited is considered to incorporate “consisting of.” Thus, an embodiment “consisting essentially of” (an) element(s) supports embodiments “consisting of” the recited element(s). Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of treating viral infection, the method comprising administering to a subject in need thereof an effective amount of an inhibitor of the EZH1 and/or EZH2 histone methyltransferase activities, wherein the viral infection is due to a herpesvirus or adenovirus or flavivirus.

2. A method of treating viral infection, the method comprising administering to a subject in need thereof an effective amount of an inhibitor of the EZH1 and/or EZH2 histone methyltransferase activities, wherein the inhibitor is a compound of Formula (I): 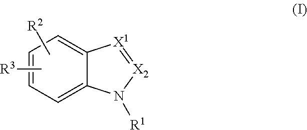
wherein
X¹ and X² are each CR⁴, X¹ is N and X² is CR⁴, or X¹ is CR⁴ and X² is N;
R¹ is alkyl optionally substituted with one or more substituents selected from cycloalkyl, heterocycloalkyl, aryl, and heteroaryl, each substituent optionally further substituted with one or more substituents selected from halo, alkyl, amino, nitro, cyano, and alkoxyl;
R² is H or —L—NR⁵—(CH₂)ₘ—X³,
L is SO₂ or CO,
m is 0 to 3,
X³ is H, cycloalkyl, heterocycloalkyl, aryl, or heteroaryl, each cycloalkyl, heterocycloalkyl, aryl, and heteroaryl optionally substituted with one or more substituents selected from halo, alkyl, amino, nitro, cyano, and alkoxyl, the cycloalkyl and heterocycloalkyl optionally having an unsubstituted methylene group replaced by CO;
R³ is H, cycloalkyl, heterocycloalkyl, aryl, or heteroaryl, each cycloalkyl, heterocycloalkyl, aryl, and heteroaryl optionally substituted with one or more substituents selected from halo, alkyl, amino, nitro, cyano, alkoxyl, cycloalkyl, heterocycloalkyl, aryl, and heteroaryl, each optional substituent cycloalkyl, heterocycloalkyl, aryl, and heteroaryl optionally further substituted with one or more substituents selected from alkyl, amino, nitro, cyano, and alkoxyl;
R⁴ is H, alkyl, or NR⁶R⁷;
R⁵ is H or alkyl;
R⁶ is H, cycloalkyl, heterocycloalkyl, aryl, or heteroaryl, each cycloalkyl, heterocycloalkyl, aryl, and heteroaryl optionally substituted with one or more substituents selected from halo, alkyl, amino, nitro, cyano, alkoxyl, cycloalkyl, heterocycloalkyl, aryl, and heteroaryl, each optional substituent alkyl, cycloalkyl, heterocycloalkyl, aryl, and heteroaryl optionally further substituted with one or more substituents selected from alkyl, amino, nitro, cyano, alkoxyl, heterocycloalkyl, aryl, and heteroaryl, each further optional substituent cycloalkyl, heterocycloalkyl, aryl, and heteroaryl optionally substituted with one or more substituents selected from alkyl, amino, nitro, cyano, and alkoxyl; and
R⁷ is H or alkyl;
or a pharmaceutically acceptable salt, solvate, or stereoisomer thereof.

3. The method of claim 2, wherein
R¹ is isopropyl, 4-fluorobenzyl, or 2-butyl;
R² is 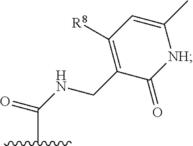
R³ is 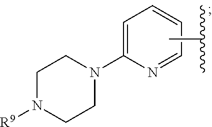
R⁴ is 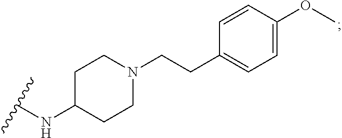 [SMILES: *NC1CCN(CCc2ccc(OC)cc2)CC1]
R⁸ is methyl or n-propyl; and
R⁹ is H, methyl, or isopropyl.

4. The method of claim 2, wherein the compound is a compound of Formula (II): 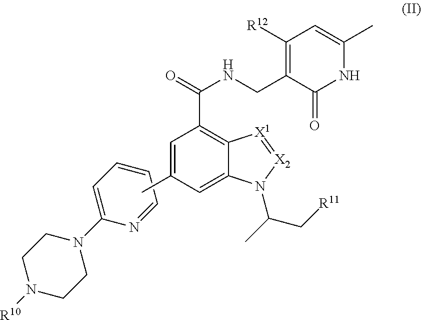
wherein X¹ and X² are each CR⁴ or X¹ is CR⁴ and X² is N; R⁴ is H or methyl; R¹⁰ is H, methyl, ethyl, or propyl; R¹¹ is H or methyl; and R¹² is methyl, ethyl, or propyl.

5. The method of claim 2, wherein the compound is

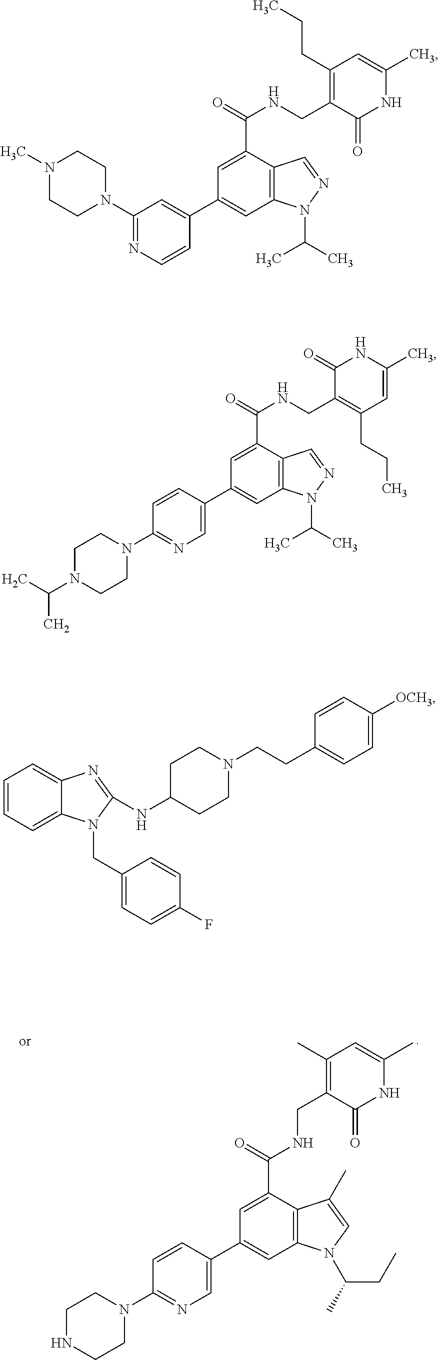 [SMILES: CCCc1cc(C)[nH]c(=O)c1CNC(=O)c1cc(-c2ccnc(N3CCN(C)CC3)c2)cc2c1cnn2C(C)C ; CCCc1cc(C)[nH]c(=O)c1CNC(=O)c1cc(-c2ccc(N3CCN(C(C)C)CC3)nc2)cc2c1cnn2C(C)C ; COc1ccc(CCN2CCC(Nc3nc4ccccc4n3Cc3ccc(F)cc3)CC2)cc1 ; CC[C@H](C)n1cc(C)c2c(C(=O)NCc3c(C)cc(C)[nH]c3=O)cc(-c3ccc(N4CCNCC4)nc3)cc21]

6. The method of claim 1, wherein the viral infection involves reactivation of a virus after latency in the subject.

7. The method of claim 1, wherein the viral infection is due to a herpesvirus or adenovirus, wherein the herpesvirus is herpes simplex type 1 and wherein the adenovirus is adenovirus 5.

8. The method of claim 1, wherein the viral infection is acute.

9. The method of claim 1, wherein the composition is a topical medicament.

10. A method of improving the therapeutic effect of a pharmaceutical composition, the method comprising adding to the pharmaceutical composition a compound of Formula (I): 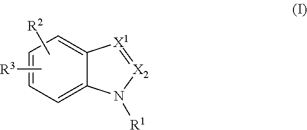
wherein
X¹ and X² are each CR⁴, X¹ is N and X² is CR⁴, or X¹ is CR⁴ and X² is N;
R¹ is alkyl optionally substituted with one or more substituents selected from cycloalkyl, heterocycloalkyl, aryl, and heteroaryl, each substituent optionally further substituted with one or more substituents selected from halo, alkyl, amino, nitro, cyano, and alkoxy;
R² is H or —L—NR⁵—(CH₂)ₘ—X³,
L is SO₂ or CO,
m is 0 to 3,
X³ is H, cycloalkyl, heterocycloalkyl, aryl, or heteroaryl, each cycloalkyl, heterocycloalkyl, aryl, and heteroaryl optionally substituted with one or more substituents selected from halo, alkyl, amino, nitro, cyano, and alkoxyl, the cycloalkyl and heterocycloalkyl optionally having an unsubstituted methylene grout replaced by CO;
R³ is H, cycloalkyl, heterocycloalkyl, aryl, or heteroaryl, each cycloalkyl, heterocycloalkyl, aryl, and heteroaryl optionally substituted with one or more substituents selected from halo, alkyl, amino, nitro, cyano, alkoxyl, cycloalkyl, heterocycloalkyl, aryl, and heteroaryl, each optional substituent cycloalkyl, heterocycloalkyl, aryl, and heteroaryl optionally further substituted with one or more substituents selected from alkyl, amino, nitro, cyano, and alkoxyl;
R⁴ is H, alkyl, or NR⁶R⁷;
R⁵ is H or alkyl;
R⁶ is H, cycloalkyl, heterocycloalkyl, aryl, or heteroaryl, each cycloalkyl, heterocycloalkyl, aryl, and heteroaryl optionally substituted with one or more substituents selected from halo, alkyl, amino, nitro, cyano, alkoxyl, cycloalkyl, heterocycloalkyl, aryl, and heteroaryl, each optional substituent cycloalkyl, heterocycloalkyl, aryl, and heteroaryl optionally further substituted with one or more substituents selected from alkyl, amino, nitro, cyano, alkoxyl, cycloalkyl, heterocycloalkyl, aryl, and heteroaryl, each further optional substituent cycloalkyl, heterocycloalkyl, aryl, and heteroaryl optionally substituted with one or more substituents selected from alkyl, amino, nitro, cyano, and alkoxyl; and
R⁷ is H or alkyl;
or a pharmaceutically acceptable salt, solvate, or stereoisomer thereof.

11. The method of claim 10,
wherein
R¹ is isopropyl, 4-fluorobenzyl, or 2-butyl;
R² is 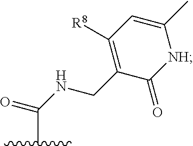
R³ is 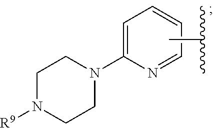
R⁴ is 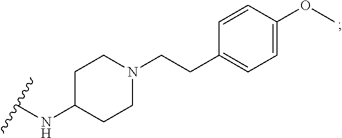 [SMILES: *NC1CCN(CCc2ccc(OC)cc2)CC1]
R⁸ is methyl or n-propyl; and
R⁹ is H, methyl, or isopropyl.

12. The method of claim 10, wherein the compound is a compound of Formula (II): 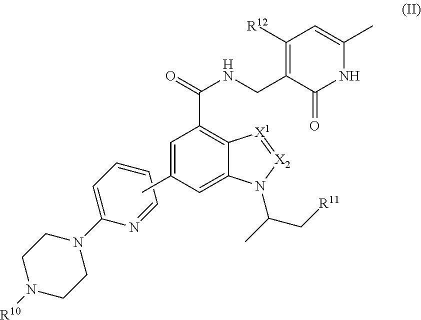
wherein X¹ and X² are each CR⁴ or X¹ is CR⁴ and X² is N; R⁴ is H or methyl; R¹⁰ is H, methyl, ethyl, or propyl; R¹¹is H or methyl; and R¹² is methyl, ethyl, or propyl.

13. The method of claim 10, wherein the compound is

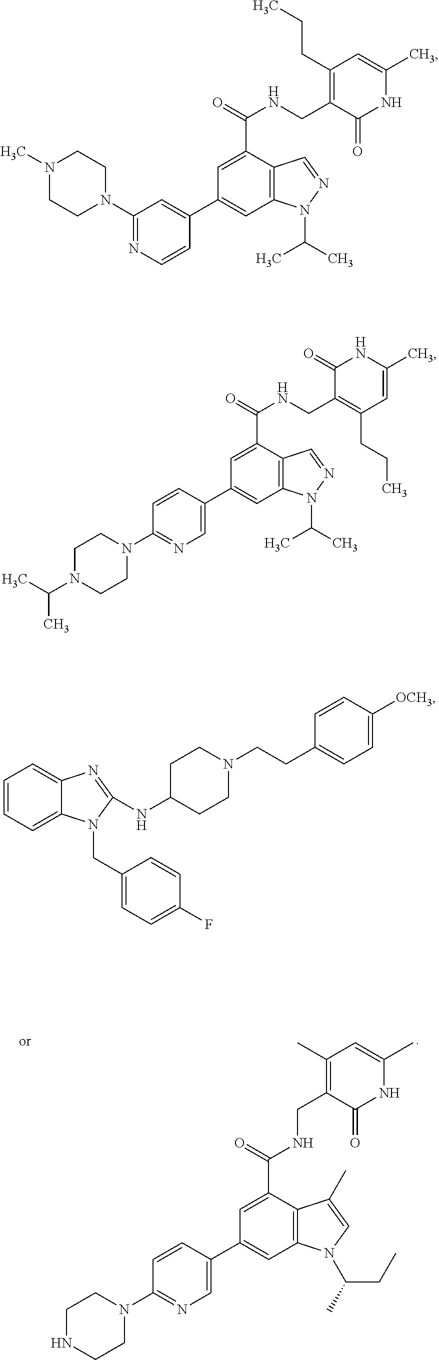 [SMILES: CCCc1cc(C)[nH]c(=O)c1CNC(=O)c1cc(-c2ccnc(N3CCN(C)CC3)c2)cc2c1cnn2C(C)C ; CCCc1cc(C)[nH]c(=O)c1CNC(=O)c1cc(-c2ccc(N3CCN(C(C)C)CC3)nc2)cc2c1cnn2C(C)C ; COc1ccc(CCN2CCC(Nc3nc4ccccc4n3Cc3ccc(F)cc3)CC2)cc1 ; CC[C@H](C)n1cc(C)c2c(C(=O)NCc3c(C)cc(C)[nH]c3=O)cc(-c3ccc(N4CCNCC4)nc3)cc21]

14. The method of claim 2, wherein the viral infection involves reactivation of the virus after latency in the subject.

15. The method of claim 2, wherein the viral infection is due to a herpesvirus or adenovirus or flavivirus.

16. The method of claim 2, wherein the viral infection is acute.

17. The method of claim 2, wherein the composition is a topical medicament.

18. The method of claim 1, wherein the compound is

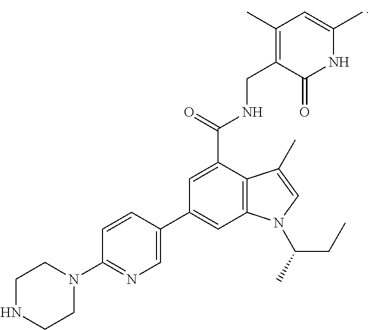 [SMILES: CC[C@H](C)n1cc(C)c2c(C(=O)NCc3c(C)cc(C)[nH]c3=O)cc(-c3ccc(N4CCNCC4)nc3)cc21]

19. The method of claim 2, wherein the compound is

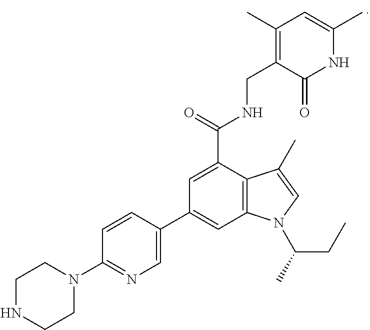 [SMILES: CC[C@H](C)n1cc(C)c2c(C(=O)NCc3c(C)cc(C)[nH]c3=O)cc(-c3ccc(N4CCNCC4)nc3)cc21]

20. The method of claim 10, wherein the compound is 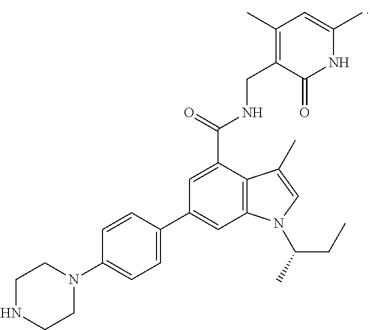 [SMILES: CC[C@H](C)n1cc(C)c2c(C(=O)NCc3c(C)cc(C)[nH]c3=O)cc(-c3ccc(N4CCNCC4)cc3)cc21]
